(19) United States
(12) Patent Application Publication
(10) Pub. No.: US 2024/0269229 A1
(43) Pub. Date: Aug. 15, 2024

(54) NOVEL LIPOPEPTIDE FORMULATION

(71) Applicant: ADVAPHARM GMBH, Moosach (DE)

(72) Inventors: Andreas SCHUTZ, Stockdorf (DE); Helmut WOLF, Moosach (DE)

(73) Assignee: ADVAPHARM GMBH, Moosach (DE)

(21) Appl. No.: 18/692,273

(22) PCT Filed: Sep. 14, 2022
(86) PCT No.: PCT/EP2022/075538

§ 371 Date: Mar. 14, 2024

(30) Foreign Application Priority Data
Sep. 14, 2021 (LU) LU500648

(51) Int. Cl. A61K 38/16; A61K 9/127; A61K 9/19; A61K 47/10; A61K 47/24; A61K 47/26; A61K 47/28

(52) U.S. Cl. CPC A61K 38/16; A61K 9/1278; A61K 9/19; A61K 47/10; A61K 47/24; A61K 47/26; A61K 47/28

(57) ABSTRACT

Novel pharmaceutical liposomal formulation of lipopeptides are provided as well as their uses and preparation.

### BACKGROUND

Bulevirtide is a novel drug candidate for treatment of chronic hepatitis B and chronic hepatitis delta (also known as hepatitis D), as well as potentially a range of inflammatory and metabolic diseases. It is disclosed in WO 2009/092612. Bulevirtide is a linear 47 amino acids chemically synthesized peptide, derived from the N-terminal domain of the large HBV surface protein (HBV=hepatitis B virus). The active substance is available as acetate salt. Its antiviral mode of action relies on specific binding and blockage of the hepatocyte surface protein NTCP. Bulevirtide-mediated inhibition of NTCP prevents entry of HBV and HDV (hepatitis D virus) into the cell.

It was recently discovered that Hepatitis B and Delta viruses enter hepatocytes through the binding to NTCP with HBV preS1 surface protein domain. As a mimetic of the preS1 domain, Myrcludex B specifically blocks the corresponding NTCP binding site, thereby inhibiting the entry of the viruses into the liver cells.

The blockade of NTCP is not only relevant to antiviral drug development. The effect of this mechanism on lipid metabolism and specifically the elevation of bile acid levels may have positive implications for the treatment of a variety of other diseases.

Hepatitis delta is the most severe form of viral hepatitis. It is caused by HDV, a small RNA virus, which requires helper functions from HBV for virion assembly and propagation and uses the HBV envelope for virus release and infection of new cells. About 5% of chronically HBV infected are co-infected with HDV.

The presence of HDV is associated with more severe and rapid progression of liver disease than HBV infection alone. Liver cirrhosis and decompensation occur earlier and more frequently in course of HBV/HDV co-infection than in HBV mono-infection.

The therapeutic options for HDV co-infected patients are very limited, since antiviral agents active against HBV do not work against HDV.

For the treatment of HDV infections, Bulevirtide, also known, e.g., as Hepcludex®, was conditionally approved as one of the first specific agents against HDV in July 2020. Bulevirtide appears as a white or off-white hygroscopic powder. It is practically insoluble in water and soluble at concentrations of 1 mg/ml in 50% acetic acid and about 7 mg/ml in carbonate buffer solution at pH 8.8, respectively. Hepcludex® is available as powder that can be stored at −20° C. or up to 3 months at a temperature between 2° C. to 8° C. according to the instruction leaflet of Hepcludex® 2 mg by MYR Pharmaceuticals. For use, Hepcludex® is to be reconstituted with water for injection purposes followed by subcutaneous injection, which is required once daily for the course of the treatment.

There are different ways to solubilize poorly soluble compounds for parenteral administration. Typical approaches are the optimization of the pH or the use of co-solvents 15 (e.g. PEG300, PEG400, propylene glycol, or ethanol). If these approaches are, for any reason, not feasible, the use of surfactants may be considered (e.g. Tween® 80 or Cremophor EL®). However, these types of surfactants are frequently associated with adverse effects.

Cyclodextrins are established as safe solubilizing agents, yet with limitations as they are not effective solubilizers for all compounds. Moreover, compounds with a high solubility in 20 natural oils (e.g. Propofol) may be solubilized in parenteral fat emulsions.

Another possibility to solubilize poorly soluble compounds is the use of phospholipids (van Hoogevest P., Xiangli L., and Alfred F. “Drug delivery strategies for poorly water-soluble drugs: the industrial perspective” Expert Opinion an Drug Delivery 2011, 8(11), 1481-1500). However, the solubilization of a certain poorly soluble compound by phospholipids cannot be predicted and usually the size of liposomes must be adopted so that the liposomes are suitable for use in a medicament.

U.S. Pat. No. 8,591,942; 9,655,846 disclosed the method of preparing liposomes containing docetaxel. In U.S. Pat. No. 8,591,942, the method comprises dispersing soy phosphatidylcholine and sodium oleate in an aqueous medium to produce dispersed liposomes.

US 2008/0166403 disclosed long circulating liposome, comprising a phospholipid bilayer and a hydrophilic core, wherein the phospholipid bilayer contains vitamin E derivative (D-alpha tocopheryl polyethylene glycol 1000 succinate, TPGS).

Usually, liposomes are prepared by dissolving lipids in organic solvents, lyophilization thereof, followed by hydration (which results in multilamellar liposomes which are usually not suitable for most applications owing to their large size and low encapsulation volumes. By using an additional step, such as extrusion or complex homogenization, the size of the large liposomes must be reduced. However, the encapsulation efficiency of these processes is usually below 70%. 50% is considered high and generally 20% to 30% encapsulation efficiencies are what can be obtained (Dan Lasic, TibTech July 1998, Vol. 16, pages 307 to 321).

In some cases encapsulation efficiencies of around 90% were observed, however, these observation referred to MLV (multilamellar vesicle) encapsulations typically having a diameter of 5 to 50 μm and result in a high and unwanted lipid to drug ratio (Liposome-Based Depot Injection Technologies Nandini V. Katre Am J Drug Deliv 2004; 2 (4): 213-227 1175-9038/04/0004-0213/$31.00/0).

WO 2014/167435 disclosed a surface functionalized liposomal formulation comprising an anticancer agent as an active ingredient, liposomes surrounded by a functional coating of D-a-Tocopheryl Polyethylene Glycol 1000 Succinate (TPGS), wherein the anticancer agent is entrapped within the liposomes, and further wherein said formulation has an encapsulation efficiency of >70%.

Muthu et al; Biomaterials. 2012 April; 33(12):3494-501 disclosed the TPGS coated liposomes for brain delivery of docetaxel prepared by solvent injection method. The reported formulations posed about 64.10+0.57% encapsulation efficiency which was significantly lower than the present invention (encapsulation efficiency˜95%).

WO 2010/078045 A2 discloses a method of preparing liposomes of constrained particle size by substantially continuously mixing substantially continuously flowing streams of water, and of an organic solvent contain lipid(s) capable of forming liposomes, and cooling the mixture so liposomes form, the ratio of the flow rate of the stream of water to the flow rate of the stream of organic solvent, and the rate of cooling of said mixture, being controlled so as to obtain a preparation of liposomes such that at least about 90% of the liposomes are of a particle size less than about 200 nm.

CN 110339166 A relates to a Liraglutide multivesicular liposome and a preparation method and an application thereof. More specifically, said liraglutide multivesicular liposome comprises Liraglutide, a membrane material, an osmotic pressure regulator and a stabilizer.

Zhang et al. (Drug Delivery 23(9):3358-3363, 2016) discloses subcutaneous Liraglutide-loaded multivesicular liposomes for treating diabetes by using a two-step water-in-oil-in-water double emulsification process.

CN 102688192 A discloses a preparation of polypeptide drug for treating diabetes and a production method thereof, wherein the preparation is mainly composed of phosphatide, sesame oil, glycerin, liraglutide, salt and ethanol.

Uhl et al. (European Journal of Pharmaceutics and biopharmaceutics, 103:159-166, 2016) discloses a liposomal formulation containing specific tetraether lipids for the oral administration of the investigational hepatitis B peptide drug Myrcludex B.

Surprisingly, it was observed that formulations of lipopeptides such as Bulevirtide or Liraglutide can be prepared with a high encapsulation efficiency by using a simple preparation method according to the invention. The preparation method according to the invention results not only in a surprisingly high load of a lipopeptide such as Bulevirtide but also in a monophasic nano-disperse system which can be freeze-dried. Rehydration of such a freeze dried monophasic nano-dispersed system leads to a liposomal formulation with a liposome size suitable for subcutaneous injections and with a favorable liposome/drug ratio.

In particular, it was surprisingly found that a monophasic nano-disperse system can easily be prepared using the method of the present invention by combining an organic phase and an aquatic phase, wherein the organic phase comprises one or more phospholipids and one or more organic solvents and wherein the one or more organic solvent forms with the aquatic phase a freezable and sublimable monophasic mixture. The said monophasic nano-disperse mixture forms (spontaneously) by simply mixing the organic phase with the aqueous phase without the need of mechanical means like high shear mixers, high pressure homogenizers or ultrasonic for reducing the size of vesicles. Said monophasic nano-disperse system comprises micelles that can be sterile filtered, without any mechanical size reduction step usually required in current approaches as listed before, and lyophilized. This is especially advantageous as such a lyophilized monophasic nano-disperse system was found to be stable and thus, can be stored and/or shipped without special temperature requirements and/or time constraints.

Moreover, lipopeptides like Bulevirtide can be stored in such a monophasic nano-disperse system prepared according to the present invention, especially well in lyophilized form, whereas said lipopeptides may not be stable in solution as in case of Bulevirtide. When required for administration, the lyophilized preparation of the monophasic nano-disperse system can be easily reconstituted by adding an aqueous solution, as e.g. typically used for reconstitution of lyophilized drugs. By reconstituting the monophasic nano-disperse system according to the present invention, preferably with an aqueous salt containing solution such as saline or buffer, liposomes are prepared in situ. In particular, larger, multilamellar liposomes can be prepared using the method of the present invention compared to current approaches, which are based on a direct preparation and mechanical size reduction of liposomes that are then sterile filtered, e.g. using pharmaceutically approved filter with 0.22 μm nominal pore size prior to freeze-drying. As multilamellar liposomes with a D90 between 1 μm and 4.5 μm can be generated in situ according to the present invention, said liposomes can be advantageously used for subcutaneously injection forming a depot which enables a prolonged release of the encapsulated drug and can also be loaded with more lipopeptides like Bulevirtide compared to liposomes generated using current methods. Of note, as regards this paragraph the same applies for a respective formulation according to the present invention.

Pharmaceutical liposomal formulations according to the present invention are particularly advantageous as regards administration of lipopeptides like Bulevirtide. Compared to subcutaneous injection of a lipopeptide like Bulevirtide in solution, administration using a pharmaceutical multilamellar liposomal formulation of the present has a slower release from the subcutaneous depot of the lipopeptide and thus, provides prolonged bioavailability. Consequently, a pharmaceutical liposomal formulation according to present invention may be required, e.g. once a week instead of daily. Thus, the pharmaceutical liposomal formulations according to the present invention, preferably comprising Bulevirtide, have the potential to significantly increase the quality of life of patients.

Hence, the present invention is related to an easy and highly efficient method of preparing a lipopeptide containing formulation based on the preparation of nano-scaled systems by combining an organic and an aqueous phase and adding lipopeptide(s). Said nano-scaled systems are thermodynamically stable and can be easily compounded, sterile filtered and filled and lyophilized according to standard procedures of pharmaceutical manufacturing. The freeze-dried preparation is characterized by high stability and shelf-life and can be easily reconstituted in situ, for example by adding water for injection or saline, to generate lipopeptide containing liposomes ready for parenteral administration, especially for subcutaneous injection.

The actual scope of the present invention is defined by the appended claims. Embodiments of the invention are the subject-matter of the dependent claims and are disclosed throughout the present description and figures.

### SUMMARY

A first aspect (Aspect 1) refers to a formulation, wherein the formulation is a monophasic nano-disperse system comprising
a) One or more phospholipid(s) selected from the group consisting of phosphatidylcholine (PC), phosphatidylinositol (PI), phosphatidylserine (PS), phosphatidylethanolamine (PE), phosphatidic acid (PA), phosphatidylglycerol (PG), or a derivative of any of the foregoing or mixtures thereof, in the range between 40% and 99.7%, preferably between 40% and 97%, more preferably between 40% and 75%, based on the total weight of a) to e);
b) a lipopeptide, preferably Bulevirtide or Liraglutide, in the range between 0.3% and 20% based on the total weight of a) to e);
c) cholesterol or a derivative thereof, preferably in the range between 0% and 14%, preferably between 4% and 14%, based on the total weight of a) to e);
d) a bulking agent, preferably glycine, arginine, proline or any other amino acid known to be suitable as a bulking agent, or a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, xylitol, xylose, dextran, or a mixture thereof, in the range between 0% and 35%, preferably between 15% and 35%, based on the total weight of a) to e);
e) a tonicity adjusting agent which is not d) in the range between 0% and 35%, preferably in the range between 0.1% and 10%, based on the total weight of a) to e);

wherein the sum of a), b), c), d) and e) always sums up to 100% and the combined amount of a) to e) is between 10% and 100% based on the total weight of the formulation. Thus, the formulation according to Aspect 1 refers to a monophasic nano-disperse system comprising micelles, loaded with a lipopeptide. Said lipopeptide is preferably Bulevirtide or Liraglutide, wherein preferably Bulevirtide is in the range between 3% and 13% based on the total weight of a) to e) or Liraglutide in the range between 0.3% and 2% based on the total weight of a) to e).

A preferred embodiment 1 refers to the formulation of Aspect 1, wherein the formulation is a lyophilized formulation. This is advantageous as a lyophilized formulation according to preferred embodiment 1 is stable and thus, can be stored and/or shipped without specific temperature requirements and/or time constraints.

A further preferred embodiment 2 refers to the lyophilized formulation according to preferred embodiment 1, wherein the amount of Bulevirtide is between 12 mg and 24 mg.

A further preferred embodiment 2 refers to the lyophilized formulation according to preferred embodiment 1, wherein the amount of Liraglutide is between 0.6 mg and 1.8 mg.

A further Aspect refers to formulation, wherein the formulation is a pharmaceutical liposomal formulation obtained by mixing/reconstituting the formulation of Aspect 1, preferred embodiment 1 and/or any of the two further preferred embodiments 2 described above herein comprising
a) one or more phospholipid selected from the group consisting of phosphatidylcholine (PC), phosphatidylinositol (PI), phosphatidylserine (PS), phosphatidylethanolamine (PE), phosphatidic acid (PA), phosphatidylglycerol (PG), or a derivative of any of the foregoing or mixtures thereof, in the range between 40% and 99.7%, preferably between 40% and 97%, more preferably between 40% and 75%, based on the total weight of a) to e);
b) a lipopeptide, preferably Bulevirtide or Liraglutide, in the range between 0.3% and 20% based on the total weight of a) to e);
c) cholesterol or a derivative thereof in the range between 0% and 14%, preferably in a range between 4% and 14%, based on the total weight of a) to e);
d) a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture thereof in the range between 0% and 35%, preferably between 15% and 35% based on the total weight of a) to e);
e) a tonicity adjusting agent which is not d) in the range between 0% and 35%, preferably in the range between 0.1% and 10%, based on the total weight of a) to e);

wherein the sum of a), b), c), d) and e) and sums up to 100% and the combined amount of a) to e) is between 10% and 50% based on the total weight of the formulation further comprising a solvent, preferably water. Preferably, the pharmaceutical liposomal formulation according to said further Aspect is obtained by mixing/reconstituting the formulation of Aspect 1, preferred embodiment 1 and/or any of the two further preferred embodiments 2 described above herein, with an aqueous solution, preferably with an aqueous salt containing solution such as saline or buffer (a physiologically acceptable solution). As regards the lipopeptide, said lipopeptide is preferably Bulevirtide or Liraglutide, wherein preferably Bulevirtide is in the range between 3% and 13% based on the total weight of a) to e) or Liraglutide in the range between 0.3% and 2% based on the total weight of a) to e).

A further preferred embodiment 4 refers to the liposomal pharmaceutical composition according to the further Aspect, wherein the tonicity adjusting agent is NaCl.

A further preferred embodiment 5 refers to the liposomal pharmaceutical formulation according to the further Aspect and/or preferred embodiment 4 further comprising a buffer system.

A further preferred embodiment 6 refers to the liposomal pharmaceutical formulation according to any one of the further Aspect and/or preferred embodiments 4 to 5 wherein the pH of the liposomal pharmaceutical formulation is between 5 and 8; preferably between 5 and 7.6, in one more preferred embodiment between 6 and 7.6 (e.g. either slightly acidic to neutral such as between 6.5 and 7 or between 7.2 and 7.6, such as between 7.3 and 7.5). A further preferred embodiment 7 refers to the formulation according to Aspect 1, the further Aspect and/or to any one of preferred embodiments 1 and 6, wherein a) is PC or a mixture of PC with one or more phospholipids selected from the group consisting of phosphatidylinositol (PI), phosphatidylserine (PS), phosphatidylethanolamine (PE) and phosphatidic acid (PA), preferably PC.

A further preferred embodiment 8 refers to the formulation according to Aspect 1, the further Aspect and/or to any one of preferred embodiments 1 to 7, wherein e) is trehalose.

A further preferred embodiment 9 refers to the formulation according to Aspect 1, the further Aspect and/or to any one of preferred embodiments 1 to 8, wherein a) is PC and a) is between 50% and 70% compared to the total sum of a), b), c), d) and e) in the formulation; b) is between 5% and 11% compared to the total sum of a), b), c), d) and e) in the formulation; d) is between 6% and 12% compared to the total sum of a), b), c), d) and e) in the formulation; and e) is trehalose and e) is between 20% and 30% compared to the total sum of a), b), c), d) and e) in the formulation, wherein the sum of a), b), c), d) and e) sums up to 100%.

A further preferred embodiment 10 refers to a pharmaceutical composition according to anyone of the further Aspect and/or preferred embodiment 4 to 9 for use as a medicament.

A further preferred embodiment 11 refers to a pharmaceutical composition for use according to preferred embodiment 10, wherein said use is in the treatment of chronic hepatitis B and/or chronic hepatitis D. This has the advantage that administration frequency of a lipopeptide like Bulevirtide can be reduced compared to administration of a respective lipopeptide in solution and thus, quality of life of a patient increased.

A further preferred embodiment 11 refers to a pharmaceutical composition for use according to preferred embodiment 10, wherein said use is in the treatment of inflammatory, preferably an inflammatory disease.

A further preferred embodiment refers to a formulation according to Aspect 1, the further Aspect and/or any of its preferred embodiments 1 to 10, comprising
a) one or more phospholipid selected from the group consisting of phosphatidylcholine (PC), phosphatidylinositol (PI), phosphatidylserine (PS), phosphatidylethanolamine (PE), phosphatidic acid (PA), phosphatidylglycerol (PG), or a derivative of any of the foregoing or mixtures thereof, in the range between 40% and 70% based on the total weight of a) to e);
b) a lipopeptide, preferably Bulevirtide or Liraglutide, in the range between 0.3% and 20% based on the total weight of a) to e);
c) cholesterol or a derivative thereof in the range between 4% and 14% based on the total weight of a) to e);
d) a bulking agent, preferably glycine, arginine, proline or any other amino acid known to be suitable as a bulking agent or a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, xylitol, xylose, dextran, or a mixture thereof, in the range between 15% and 35% based on the total weight of a) to e);
e) a tonicity adjusting agent which is not d) in the range between 0% and 35% based on the total weight of a) to e);

wherein the sum of a), b), c), d) and e) sums up to 100%.

In one preferred embodiment, a formulation according to Aspect 1 or any of its preferred embodiments 1 or 2, wherein the formulation further comprises tert-butanol in a range between 0.01% and 2% based on the total weight of the formulation.

In yet another preferred embodiment, a formulation according to Aspect 1 or any of its preferred embodiments 1 or 2, wherein the formulation further comprises tert-butanol in a range between 0.01% and 2% based on the total weight of the formulation and the combined amount of a) to e) is between 98% and 99.99% based on the total weight of the formulation. A further Aspect 2 refers to the use of a formulation according to Aspect 1 or any one of preferred embodiment 1 or 2, for the preparation of a medicament to treat chronic hepatitis B and/or chronic hepatitis D or inflammatory, preferably for the preparation of a medicament to treat chronic hepatitis B and/or chronic hepatitis D or an inflammatory disease.

A further Aspect 3 refers to a kit comprising a formulation according to Aspect 1 or any one of preferred embodiment 1 or 2 and separated a pharmaceutical aqueous solution. E.g., a kit comprising a formulation according to Aspect 1 or any one of preferred embodiment 1 or 2 in a container and a pharmaceutical aqueous solution in a second container. Optionally, further comprising instructions for mixing the two components of the two containers to receive a, preferably ready-to-use, pharmaceutical liposomal formulation. Thus, Aspect 3 preferably relates to a kit comprising a lyophilized monophasic nano-disperse system, loaded with a lipopeptide, and a pharmaceutical aqueous solution that can be used to reconstitute the lyophilized monophasic nano-disperse system, thus preparing liposomes loaded with said lipopeptide for subsequent use, preferably for subsequent subcutaneous injection.

A further Aspect 4 refers to a method for preparing a formulation comprising the steps of
i) providing an organic phase comprising
   one or more phospholipid(s) selected from the group consisting of phosphatidylcholine (PC), phosphatidylinositol (PI), phosphatidylserine (PS), phosphatidylethanolamine (PE), phosphatidic acid (PA), phosphatidylglycerol (PG), or a derivative of any of the foregoing or a combination of any of the foregoing;
   optionally Cholesterol or a derivative of Cholesterol; and
   at least one organic solvent;
ii) providing an aqueous phase comprising an aqueous medium; and
   optionally a bulking agent selected from the group consisting of glycine, arginine, proline or any other amino acid known to be suitable as a bulking agent, a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, xylitol, xylose, dextran, or a mixture of any of the foregoing,
   optionally a pharmaceutically acceptable buffer, and
   optionally a pharmaceutically acceptable tonicity adjusting agent which is not a bulking agent selected from the group consisting of glycine, arginine, proline or any other amino acid known to be suitable as a bulking agent, a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture of any of the foregoing,
   wherein the pH of the aqueous phase is between 3 and 9, e.g., between 5.5 and 7, such as between 5.8 and 6.7 (for example, by using a sodium acetate buffer);
iii) Combining the organic phase and the aqueous phase, wherein the mixing ratio of the organic phase and the aqueous phase is between 10:1 (v/v) and 1:10 (v/v), preferably between 2:1 (v/v) and 1:4(v/v), more preferably between 1.5:1 (v/v) and 1:4 (v/v) such as between 1:1 (v/v) to 1:3 (v/v), around 1+0.5:1+0.5 (v/v), around 1+0.5:2+0.5 (v/v) or around 1+0.5:3+0.5 (v/v), e.g. 1:1 (v/v), 1:2 (v/v) or 1:3 (v/v), resulting in combined organic and aqueous phases; wherein said at least one organic solvent and the aqueous phase form a monophasic mixture, preferably a freezable and sublimable monophasic mixture;
iv) adding a lipopeptide, preferably Bulevirtide or Liraglutide, more preferably Bulevirtide, either to the organic phase of step i) and then mixing the organic phase with the lipopeptide with the aqueous phase as described in step iii), or to the aqueous phase of step ii) and then mixing the aqueous phase with the lipopeptide with the organic phase as described in step iii), or to the combined phases of step iii), preferably to the combined phases of step iii), resulting in a lipopeptide formulation, wherein said lipopeptide formulation so obtained is a monophasic nano-disperse system.

Hence, it is envisioned that a monophasic mixture is formed by combining the aqueous phase and the at least one organic solvent comprised in the organic phase, wherein said monophasic mixture is preferably freezable and sublimable. This has the advantage that the organic phase can substantially be removed from the formulation by lyophilisation. Herein, the term “freezable” refers to the physiochemical property of a mixture to form a solid matrix below a temperature in the range between 0° C. and −60° C. In particular, it is preferred that at a temperature in the range between 0° C. and −60° C. the monophasic mixture prepared in step iii) is substantially forming a solid matrix. Furthermore, the term “sublimable” is intended to be understood as the physiochemical property of a mixture to transfer from a solid to a gas state without any intermediate liquid state at a pressure in the range between 1 Pa and 100 Pa and at a temperature in the range between 0° and −60° C.

Hence, a preferred monophasic mixture can be obtained by combining the aqueous phase, preferably water, and the organic phase comprising at least one organic solvent, wherein said at least one organic solvent is preferably selected from the group consisting of anisole, ethylacetate, 1,4-dioxane, dimethylcarbonate, dimethylsulfoxide, glycofurol, N,N-dimethylacetamide, N,N-dimethylformamide, N-methyl-2-pyrrolidone (NMP), isopropylideneglycerol, an alcohol, preferably an alcohol selected from the group consisting of 1-butanol, 2-butanol and tert-butanol, acetic acid, ethyl lactate (ethyl 2-hydroxypropanoate), acetonitrile, and a combination of any of the foregoing.

Alternatively or optionally, the at least one organic solvent may be selected from the group consisting of anisole, ethylacetate, 1,4-dioxane, dimethylcarbonate, dimethylsulfoxide, glycofurol, N,N-dimethylacetamide, N,N-dimethylformamide, N-methyl-2-pyrrolidone (NMP), isopropylideneglycerol, 1-butanol, 2-butanol and tert-butanol and a combination of any of the foregoing.

Alternatively or optionally, it is particularly preferred that the at least one organic solvent is selected from the group consisting of an alcohol, preferably tert-butanol, anisole (phenoxymethane), dimethylsulfoxide, 1,4-dioxane and dimethylcarbonate and a combination thereof. Preferably, the at least one organic solvent comprises tert-butanol or is tert-butanol.

Alternatively or optionally, the at least one organic solvent may be selected from the group consisting of acetic acid, ethyl lactate (ethyl 2-hydroxypropanoate) and acetonitrile.

It should be understood that the listed examples of organic solvents are given for purposes of illustration and not by way of limitation. In particular, the skilled person is aware how to identify suitable organic solvents and thus, organic solvents able to form a, preferably freezable and sublimable, monophasic mixture with an aqueous solution like water.

It has surprisingly found that preparing a monophasic mixture according to the method of the invention has the advantageous effect that the one or more phopsholipids comprised in the organic phase build a monophasic nano-disperse system that can be sterile filtered.

In one preferred embodiment, the method according to Aspect 4 comprises the steps of
i) providing an organic phase comprising
   one or more phospholipid(s) selected from the group consisting of phosphatidylcholine (PC), phosphatidylinositol (PI), phosphatidylserine (PS), phosphatidylethanolamine (PE), phosphatidic acid (PA), phosphatidylglycerol (PG), or a derivative of any of the foregoing or a combination of any of the foregoing;
   optionally Cholesterol or a derivative of Cholesterol;
   and at least one organic solvent selected from the group consisting anisole, ethylacetate, 1,4-dioxane, dimethylcarbonate, dimethylsulfoxide, glycofurol,
   N,N-dimethylacetamide, N,N-dimethylformamide, N-methyl-2-pyrrolidone (NMP), isopropylideneglycerol, an alcohol, preferably an alcohol selected group consisting of 1-butanol, 2-butanol and tert-butanol, acetic acid, ethyl lactate (ethyl 2-hydroxypropanoate), acetonitrile, or a combination of any of the foregoing, preferably the at least one organic solvent is selected from the group consisting of an alcohol, preferably tert-butanol, anisole (phenoxymethane), dimethylsulfoxide, 1,4-dioxane and dimethylcarbonate and a combination thereof;
ii) providing an aqueous phase comprising an aqueous medium; and
   optionally a pharmaceutically acceptable buffer, and optionally a bulking agent selected from the group consisting of glycine, arginine, proline or any other amino acid known to be suitable as a bulking agent, a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, xylitol, xylose, dextran, or a mixture of any of the foregoing,
   optionally a pharmaceutically acceptable tonicity adjusting agent which is not a bulking agent selected from the group consisting of glycine, arginine, proline or any other amino acid known to be suitable as a bulking agent, a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture of any of the foregoing,
   wherein the pH of the aqueous phase is between 3 and 9, e.g., between 5.5 and 7, such as between 5.8 and 6.7 (for example, by using a sodium acetate buffer);
iii) Combining the organic phase and the aqueous phase, wherein the mixing ratio of the organic phase and the aqueous phase is between 10:1 (v/v) and 1:10 (v/v), more preferably between 2:1 (v/v) and 1:4(v/v), even more preferably between 1,5:1 (v/v) and 1:4 (v/v) such as between 1:1 (v/v) to 1:3 (v/v), around 1±0.5:1±0.5 (v/v), around 1±0.5:2±0.5 (v/v) or around 1±0.5:3±0.5 (v/v), e.g. 1:1 (v/v), 1:2 (v/v) or 1:3 (v/v), resulting in combined organic and aqueous phases; wherein said at least one organic solvent and the aqueous phase form a monophasic mixture, preferably a freezable and sublimable monophasic mixture;
iv) adding a lipopeptide, preferably Bulevirtide or Liraglutide, more preferably Bulevirtide either to the organic phase of step i) and then mixing the organic phase with the lipopeptide with the aqueous phase as described in step iii), or to the aqueous phase of step ii) and then mixing the aqueous phase with the lipopeptide with the organic phase as described in step iii), or to the combined phases of step iii), preferably to the combined phases of step iii), resulting in a lipopeptide formulation, wherein said lipopeptide formulation so obtained is a monophasic nano-disperse system.

One preferred embodiment 1 of Aspect 4 and the preferred embodiment described above refers to the method, wherein the organic solvent is tert-butanol.

One preferred embodiment 2 of Aspect 4 and its preferred embodiments refers to the method, wherein the lipopeptide is Bulevirtide or Liraglutide.

One preferred embodiment 3 of Aspect 4 and its preferred embodiments refers to the method, wherein the phospholipid comprises PC.

One preferred embodiment 4 of Aspect 4 and its preferred embodiments refers to the method, wherein the pH of the aqueous phase is between 5 and 6.

One preferred embodiment 5 of Aspect 4 and its preferred embodiments refers to the method further comprising a step v) of lyophilizing the formulation (monophasic nano-disperse system) resulting from step iv) resulting in a lyophilizate. It is to be understood that said step v) is a preferred, though purely optional step that is termed “step v)” for clarification purposes only.

One preferred embodiment 6 of Aspect 4 and preferred embodiment 5 refers to the method further comprising a step of rehydrating the lyophilizate obtained in step v) with an aqueous solution resulting in a liposomal formulation. In particular, said preferred embodiment 6 relates thus to a rehydration step of mixing the lyophilizate obtained in step v) with an aqueous solution resulting in a liposomal formulation. Said liposomal formulation is a reconstituted liposomal formulation. Herein, the term “reconstitution” refers to rehydration of a lyophilized formulation (monophasic nano-disperse system) by mixing the lyophilizate with an aqueous solution, preferably with saline or water, more preferably with saline.

One preferred embodiment 7 of Aspect 4 and preferred embodiment 6 refers to the method, wherein the D90 of the liposomes of the liposomal formulation resulting from the rehydration step (reconstitution) is between 1 μm and 4.5 μm, preferably below 2.5 μm, more preferably between 0.025 μm and 2.5 μm, even more preferably between 0.1 μm and 2 μm. Thus, a liposomal formulation can be prepared by mixing/reconstituting a lyophilized monophasic nano-disperse system with an aqueous solution, preferably with saline or water, more preferably with saline.

One preferred embodiment 8 of Aspect 4 and its preferred embodiments 6 and 7, wherein the aqueous solution comprises at least 95% water.

One preferred embodiment 9 of Aspect 4 and its preferred embodiments 6 to 8 refers to the method, wherein the aqueous solution used for mixing/reconstituting is an aqueous NaCl solution wherein the amount of NaCl is between 8 g/l and 10 g/l, preferably between 8.8 g/l and 9.2 g/l, more preferably 9 g/l.

One preferred embodiment 10 of Aspect 4 refers to the method according to Aspect 4 and its preferred embodiments 2-4 or preferred embodiments 6-9, wherein the lipopeptide formulation or the liposomal formulation is a pharmaceutical formulation.

One preferred embodiment 11 of Aspect 4 refers to the method according to Aspect 4 and its preferred embodiments 2-4 or 10, wherein the D90 is less than 60 nm, preferably less than 25 nm, more preferably 20 nm or less, even more preferably between 3 nm and 20 nm. In one more preferred embodiment, the D90 is between 10 nm and 20 nm, in another more preferred embodiment, the D90 is between 3 nm and 5 nm.

Another Aspect 5 refers to a formulation prepared according to a method according to Aspect 4 and its preferred embodiments disclosed herein.

In one preferred embodiment of Aspect 5, the formulation is a pharmaceutical formulation.

### Definitions

If not stated otherwise, amounts in % refer to % (weight/weight) ((w/w)).

If not explicitly mentioned otherwise (e.g. by using a term such as “a specific” meaning “one”), the term “a” is an indefinite article encompassing “one” and “one or more”/“more than one” noun(s) following the term “a”.

A “buffer” or “buffer system” as used herein is used to prevent changes in the pH of a solution, and suitable examples are well-known to the skilled formulator.

A “bulking agent” as used herein and as the name implies, form the bulk of a lyophilized product, and provide an adequate structure to a lyophilized cake. Non limiting examples of bulk agents are mannitol, glycine, arginine, proline, glucose, sucrose, lactose, trehalose, and dextran.

The term “Cholesterol” refers to 3β-Hydroxy-5-cholestene (CAS No.: 57-88-5). Examples of derivatives of cholesterol are cholesteryl sulfate and its salts (e.g., sodium salt), cholesteryl hemisuccinate, cholesteryl succinate, cholesteryl oleate, polyethylene glycol derivatives of cholesterol (cholesterol-PEG), coprostanol, cholestanol, cholestane, cholic acid, cortisol, corticosterone, hydrocortisone and calciferol. Thus, a derivative of cholesterol is preferably selected from the group consisting of cholesteryl sulfate, a salt of cholesteryl sulfate, cholesteryl hemisuccinate, cholesteryl succinate, cholesteryl oleate, cholesterol-PEG, coprostanol, cholestanol, cholestane, cholic acid, cortisol, corticosterone, hydrocortisone and calciferol.

The term “Container” as used herein means an ampoule or vial with rubber stopper and cap, single or double chamber syringe, infusion bag or bottle made from polymeric materials or glass, suitable for housing compositions for parenteral administration. It also includes any vessel for holding liquids.

The term “D90” is well known to a skilled person and refers in connection with size distributions to the amount of vesicles with diameters at or below a given value having a weight of 90% of the weight of the components forming such particles in a formulation. The D90 can be determined via multiangle light scattering (MALS).

The term “in the range between X % and Y %” (wherein X and Y represent any number between 0 and 100 and X % is smaller than Y %) refers to any number within the range between X % and Y % including X % and Y %.

A “nano-disperse system” as used herein refers to an aqueous formulation comprising vesicles with a D90 of 60 nm or less, wherein the vesicles are no liposomes. More specifically, said vesicles can be seen as precursors of liposomes as liposomes can be generated by reconstituting/mixing a lyophilizate of said aqueous formulation comprising vesicles, preferably reconstitution using saline or water, more preferably using saline. Said vesicles are referred herein also as micelles in the context of a monophasic nano-disperse system. It is noted here that the inventors have found that the monophasic nano-disperse system according to the invention has liquid-crystalline properties, (spontaneously) forming nano-scale self-assembled structures analogous to micelles in case of lyotropic liquid crystals (see in this context also the English Wikipedia entry on lyotropic liquid crystals last edited on 12 Sep. 2022). Herein, the terms “nano-disperse system” and “nano-dispersed system” are used interchangeably herein. Further herein, a nano-disperse systems is a monophasic nano-disperse system.

A “lipopeptide” as a peptide, wherein a lipid chain is covalently bound to the peptide. It has been found that such a modification of a peptide, especially if the peptide has less than 50 amino acids, has been found to inhibit proteolytic attack due to the lipid chain non-covalently interacting with, e.g., serum albumin to increase the molecular weight, thus reducing, e.g., renal filtration when administered to a patient. Typically, there are three types of lipidation, and they differ based on the bond formation methods between the lipid and the peptide: amidation, esterification (S— or O—) and S-bond (ether or disulfide) formation.

Amidation and O-esterification form strong covalent bonds that are irreversible, whereas the other two methods are weak and reversible covalent bonds. The method used, as well as the lipid chain, position of lipidation, and the spacer used, all have significant impacts on physiochemical properties and bioactivity (Zhang and Bulaj “Converting Peptides into Drug Leads by Lipidation”. Current Medicinal Chemistry, Vol 19, Issue 11, 2012).

A “therapeutic lipopeptide” is a peptide or polypeptide (oligomers) that is used for the treatment of diseases. Naturally occurring peptides may serve as hormones, growth factors, neurotransmitters, ion channel ligands, and anti-infectives; lipopeptide therapeutics mimic such functions. Lipopeptide therapeutics are usually relatively safe and well-tolerated as peptides can be metabolized by the body.

Liraglutide (CAS No: 204656-20-2) (γ-L-Glutamoyl(N-α-hexadecanoyl)-Lys²⁶, Arg³⁴-GLP-1(7-37) also known as N²⁶-(Hexadecanoyl-gamma-glutamyl)-[34-arginine]GLP-1-(7-37)-peptide (WHO) or NN 2211) is a lipopeptide with 31 amino acids. Liraglutide is an anti-diabetic medication used to treat type 2 diabetes, obesity, and chronic weight management. Thus, it is also a therapeutic lipopeptide.

Bulevirtide (CAS No: 2012558-47-1) is a lipopeptide with 47-amino acid peptide with a fatty acid, a myristoyl residue, at the N-terminus and an amidated C-terminus. The active substance is available as acetate salt. The counter ion acetate is bound in ionic form to basic groups of the peptide molecule and is present in a non-stoichiometric ratio. The chemical name of Bulevirtide is (N-Myristoyl-glycyl-L-threonyl-L-asparaginyl-L-leucyl-L-seryl-L-valyl-Lprolyl-L-asparaginyl-L-prolyl-L-leucyl-glycyl-L-phenylalanyl-L-phenylalanyl-L-prolyl-L-aspartyl-L-histidyl-Lglutaminyl-L-leucyl-L-aspartyl-L-prolyl-L-alanyl-L-phenylalanyl-glycyl-L-alanyl-L-asparaginyl-L-seryl-Lasparaginyl-L-asparaginyl-Lprolyl-L-aspartyl-L-tryptophanyl-L-aspartyl-L-phenylalanyl-L-asparaginyl-L-prolyIL-asparaginyl-L-lysyl-L-aspartyl-L-histidyl-L-tryptophanyl-L-prolyl-L-glutamyl-L-alanyl-L-asparaginyl-L-lysyIL-valylglycinamide, acetate salt. Bulevirtide is an antiviral medication for the treatment of chronic hepatitis D. It is administered through subcutaneous injection. Thus, it is also a therapeutic lipopeptide. It is usually sold in the form of its acetate salt.

A “liposome” is a spherical vesicle having at least one lipid bilayer. The liposome can be used as a vehicle for administration of pharmaceutical drugs. Liposomes are most often composed of phospholipids, especially phosphatidylcholine, but may also include other lipids, such as egg phosphatidylethanolamine, so long as they are compatible with lipid bilayer structure.

“Liposomal formulation” as used herein means a liquid comprising liposomes, said liposomes comprising phospholipids. Said liposomal formulation is suitable for solubilizing a lipopeptide such as Bulevirtide or Liraglutide in an aqueous environment.

Liposomal size or vesicle size (such as micelle size), respectively, as disclosed herein refers to the size as determined by multiangle light scattering (MALS) or alternatively by dynamic light scattering (DLS). Usually, the size of liposomes is in the range from 0,025 μm to 2.5 μm (Akbarzadehet al. Nanoscale Research Letters 2013,8:102).

“Loading” means incorporating or transferring Bulevirtide into liposomes/encapsulating Bulevirtide with the liposomes.

“Pharmaceutically acceptable” means approved or approvable by a regulatory agency in a country or that is listed in the European or U.S. Pharmacopoeia or other generally recognized pharmacopoeia for use in animals, and more particularly, in humans.

PEG means polyethylene glycol.

PEGylation is the process of both covalent and non-covalent attachment or amalgamation of PEG polymer chains to molecules and macrostructures, such as phospholipids, which leads to vesicles then described as PEGylated. PEGylated phospholipids are well known and commercially available.

The “pharmaceutical composition” described herein is, in particular, a pharmaceutical liposomal composition. A “pharmaceutical liposomal composition” means a composition comprising liposomes which is suitable for pharmaceutical administration.

The phospholipids used in the formulations of the present invention can be selected form the group consisting of a natural phospholipid, a synthetic phospholipid, and combinations thereof. Lecithin is one of the natural resources for phospholipid. Lecithin is a mixture found in egg yolk and soya. It comprises a number of phospholipids including phosphatidylcholine (PC), phosphatidylethanolamine (PE), and phosphatidylinositol (PI) or a (pharmaceutically acceptable) salt of any of the foregoing.

Generally, the structure of a phospholipid as used herein is a phospholipid of structure (I)

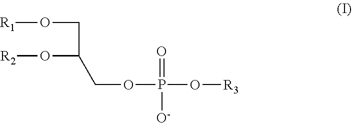

wherein

R₁ represents C₁₀-C₂₄acyl:

R₂ represents C₁₀-C₂₄acyl or hydrogen;

R₃ represents 2-trimethylamino-1-ethyl (resulting in PC), 2-amino-2-carboxy-1ethyl (resulting in PS), inosityl group (C₆H₁₁O₅) (resulting in PI), 2-amino-1ethyl (resulting in PE) or hydrogen (resulting in PA).

The terms “phosphatidic acid” and “PA” are used interchangeably herein. PA or a (pharmaceutically acceptable) salt thereof can derive from a natural source and/or a synthetic source. Non-limiting examples for PA are DLPA, DMPA, DPPA, DSPA, POPA, POPA, DEPA, HSPA, HEPA or a (pharmaceutically acceptable) salt of any of the foregoing.

The terms “phosphatidylcholine” and “PC” are used interchangeably herein. PC or a (pharmaceutically acceptable) salt thereof can derive from a natural source and/or a synthetic source. PC can be PEGylated. Non-limiting examples for PC are dilauroylphosphatidylcholine, (DLPC), dimyristoylphosphatidylcholine (DMPC), dipalmitoylphosphatidylcholine (DPPC), distearoylphosphatidylcholine (DSPC), dioleoylphosphatidylcholine (DOPC), palmitoyl-oleoyl-phosphatidylcholine (POPC), dierucoylglycerophosphocholine (DEPC), hydrogenated soy phosphatidylcholine (HSPC), hydrogenated egg phosphatidylcholine (HEPC) or a (pharmaceutically acceptable) salt of any of the foregoing.

The terms “phosphoethanolamine” and “PE” are used interchangeably herein. PE can be PEGylated. PE or a (pharmaceutically acceptable) salt thereof can derive from a natural source and/or a synthetic source. Non-limiting examples PE are DLPE, DMPE, DPPE, DSPE, POPE, POPE, DEPE, HSPE, HEPE or a (pharmaceutically acceptable) salt of any of the foregoing.

The terms “phosphatidylglycerol” and “PG” are used interchangeably herein. PG or a (pharmaceutically acceptable) salt thereof can derive from a natural source and/or a synthetic source. Non-limiting examples for PG are DLPG, DMPG, DPPG, DSPG, POPG, POPG, DEPG, HSPG, HEPG or a (pharmaceutically acceptable) salt of any of the foregoing.

The terms “phosphatidylinositol” and “PI” are used interchangeably herein. PI can be PEGylated. PI or a (pharmaceutically acceptable) salt thereof can derive from a natural source and/or a synthetic source. Non-limiting examples for PI are DLPI, DMPI, DPPI, DSPI, POPI, POPI, DEPI, HSPI, HEPI or a (pharmaceutically acceptable) salt of any of the foregoing.

The terms “phosphoserine” and “PS” are used interchangeably herein. PS can be PEGylated. PS or a (pharmaceutically acceptable) salt thereof can derive from a natural source and/or a synthetic source. Non-limiting examples for PS are DLPS, DMPS, DPPS, DSPS, POPS, POPS, DEPS, HSPS, HEPS or a (pharmaceutically acceptable) salt of any of the foregoing.

Non-limiting examples for pharmaceutically acceptable salts of any of the phospholipids are sodium salts or ammonium salts such as PG-Na PG-NH₄, DSPG-Na or DSPG-NH₄.

Thus, a derivative of any of the phospholipid(s) selected from the group consisting of phosphatidylcholine (PC), phosphatidylinositol (PI), phosphatidylserine (PS), phosphatidylethanolamine (PE), phosphatidic acid (PA), and phosphatidylglycerol (PG) is selected from the group consisting of DLPA, DMPA, DPPA, DSPA, POPA, POPA, DEPA, HSPA, HEPA, DLPC, DMPC, DPPC, DSPC, DOPC, POPC, DEPC, HSPC, HEPC, DLPE, DMPE, DPPE, DSPE, POPE, POPE, DEPE, HSPE, HEPE, DLPG, DMPG, DPPG, DSPG, POPG, POPG, DEPG, HSPG, HEPG, DLPI, DMPI, DPPI, DSPI, POPI, POPI, DEPI, HSPI, HEPI, DLPS, DMPS, DPPS, DSPS, POPS, POPS, DEPS, HSPS, HEPS, a PEGylated form of any of the foregoing and a salt of any of the foregoing.

Mixing “shortly before administration to patient” means up to three days before, in particular up to 24 hours before, and for example up to 6 hours before administration to the patient.

A “tonicity adjusting agent” means a pharmaceutically acceptable compound which can be added to a formulation to make it isotonic with human plasma. Tonicity adjusting agents include for example dextrose, glucose, mannitol, sucrose, lactose, trehalose, glycerin and NaCl, particularly sucrose or glycerin or NaCl, more particularly sucrose or NaCl. Tonicity is the ‘effective osmolality’ and is equal to the sum of the concentrations of the solutes which have the capacity to exert an osmotic force across the membrane. Parenteral formulations should be isotonic with blood plasma. Tonicity adjusting agents are well known to the skilled person.

As used herein, the terms “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment, “treat”, “treating” or “treatment” refer to alleviating or ameliorating at least one physical parameter including those which may not be discernible by the patient. In yet another embodiment, “treat”, “treating” or “treatment” refer to modulating the disease or disorder, either physically, (e.g., stabilization of a discernible symptom), physiologically, (e.g., stabilization of a physical parameter), or both.

The term “wherein the sum of a), b), c), d) and e) sums up to 100%” means that the percent values given to the components a) to e) always have to be chosen in a way that the sum of the values for a) to e) will be 100. Thus, these values give a ratio between the components a) to e). However, the skilled person understands that a formulation may comprise additional components such as a solvent. However, the amount of such an additional component is not considered when calculating the ratios between a) to e) (which values in % always have to sum up to 100%).

It is understood that, as long as a combination does not violate any law of nature, all embodiments of the present invention, irrespective of the fact if an embodiment is an embodiment, a preferred embodiment, a more preferred embodiment, a particularly preferred embodiment or a most preferred embodiment, can be combined and such combinations of two or more embodiments of the invention are disclosed herein by disclosing the two or more embodiments even if the combination of such two or more embodiments is not explicitly referred to.

### DETAILED DESCRIPTION

The present invention refers to a simple method for the preparation of formulation comprising a lipopeptide. The methods of the invention allow the easy preparation of a liquid formulation (lipopeptide formulation) wherein the D90 of the vesicles, preferably micelles, is 60 nm or less, more preferably 25 nm or less, preferably 20 nm or less. This monophasic nano-dispersed system which is formed during a method according to the invention allows the preparation of a liposomal formulation without the need to mechanically reducing the size of liposomes for the final liposomal formulation.

Alternatively, the methods of the invention further allow the easy preparation of a liposomal formulation, wherein the D90 of the liposomes is between 1 μm and 4.5 μm. The present invention further provides a formulation comprising a lipopeptide prepared according to a method of the present invention and more specifically a formulation of a lipopeptide, preferably a therapeutic lipopeptide, more preferably Bulevirtide or Liraglutide, suitable for parenteral administration to patients. In particular, such administration is by intravenous injection or infusion. The invention further provides two separate formulations which can be mixed together shortly before administration to the patient, in order to provide the liposomal composition suitable for administration. One formulation may be a lyophilizate comprising a lipopeptide, the second formulation may be an aqueous formulation. When the two separate formulations are mixed together, Bulevirtide is loaded into the forming liposomes, enabling solubilization of Bulevirtide, and resulting in a pharmaceutical liposomal composition suitable for use in the clinic.

The formulation comprising a, preferably therapeutic, lipopeptide should enable efficient and optimal loading of said therapeutic lipopeptide into liposomes before administration to the patient.

Preferably, the invention provides a pharmaceutical liposomal formulation which enables a fast release of Bulevirtide from the liposomes after administration (injection).

Overall, the invention described herein enables effective administration of a lipopeptide, preferably a therapeutic lipopeptide (e.g. Bulevirtide or Liraglutide) to patients, despite the challenging chemical characteristics of the drugs.

The formulations described herein are, in particular, pharmaceutical formulations, such as a pharmaceutical liposomal composition.

Preferably, the D90 of liposomes of a liposomal formulation is between 1 μm and 4.5 μm.

In particular, a phospholipid described herein is selected from egg lecithin, soy lecithin, or synthetic phospholipids.

Described below are a number of aspects and embodiments of the invention.

### Formulation

A first aspect refers to a formulation, wherein the formulation is a monophasic nano-disperse system comprising
a) one or more phospholipid selected from the group consisting of phosphatidylcholine (PC), phosphatidylinositol (PI), phosphatidylserine (PS), phosphatidylethanolamine (PE), phosphatidic acid (PA), phosphatidylglycerol (PG), or a derivative of any of the foregoing or mixtures thereof, in the range between 40% and 99.7%, preferably in the range between 40% and 97%, based on the total weight of a) to e), e.g., around 60%;
b) a lipopeptide, preferably a therapeutic lipopeptide, more preferably Bulevirtide or Liraglutide, in the range between 0.3% and 20% based on the total weight of a) to e), preferably in the range between 3% and 18%, more preferably in the range of 3% and 13%, such as around 8% when the lipopeptide is Bulevirtide or in the range between 0.3% and 2% when the lipopeptide is Liraglutide, such as 0.37+0.05% (i.e. between 0.365% to 0.375%), 0.6+0.05%, 0.7+0.05%, 1.1±0.06% 1.2±0.06% 1.8+0.06%;
c) cholesterol or a derivative thereof in the range between 0% and 14%, preferably between 4% and 14%, based on the total weight of a) to e), e.g., around 9%;
d) a bulking agent, preferably glycine, arginine, proline or any other amino acid known to be suitable as a bulking agent, or a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture thereof, more preferably glycine, arginine, proline, or a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture thereof, even more preferably glycine, arginine, proline, mannitol, glucose, sucrose, lactose, trehalose or dextran, even more preferably mannitol, glycine or trehalose, in the range between 0% and 35%, preferably between 15% and 35% based on the total weight of a) to e, e.g., around 25%;
e) a tonicity adjusting agent which is not d) in the range between 0% and 35% based on the total weight of a) to e);

wherein the sum of a), b), c), d) and e) sums up to 100% and the combined amount of a) to e) is between 10% and 100% based on the total weight of the formulation.

In regard of c) and e), these components are in some formulations optionally which is demonstrated by the range which has 0% as lowest boarder.

The skilled person will understand that traces of solvents and additives used in the preparation method, preferably in a preparation method of the invention described further below (such as tert-butanol, or water, or a buffer system), can still be present up to an amount of up to 2% when a formulation consists of a), b), d), optionally c), and optionally e).

One preferred embodiment refers to a formulation according to the invention, wherein the formulation consists of a), b), c), d), and e), and a residues of a solvent, preferably tert-butanol, wherein the amount of tert-butanol is between 0.01% and 2% based on the total weight of such a formulation (e.g. the combined amount of a) to e) and tert-butanol, e.g. in case of a lyophilizate). More preferably, the amount of tert-butanol is 1% or less, such as between 0.001% and 0.8%.

Another preferred embodiment refers to a formulation according to the invention, wherein the formulation consists of a), b), c), d), and e), tert-butanol and a buffer system, preferably an acetate buffer system, wherein the combined amount of tert-butanol and the buffer system is between 0.01% and 2% based on the total weight of such a formulation (e.g. the combined amount of a) to e) and tert-butanol, e.g. in case of a lyophilizate).

In yet another preferred embodiment, the formulation according to the invention comprises a liposome, proliposome, lipid clathrate, lipid colloidal dispersion, micelle, inverted micelle, discoid structure, or a combination thereof.

One further preferred embodiment refers to a, preferably pharmaceutical, monophasic nano-disperse system, wherein the D90 of the vesicles is 60 nm or less, preferably less than 25 nm, more preferably 20 nm or less, such as between 10 nm and 20 nm e.g., around 15 nm, or between 3 nm and 10 nm, e.g., around 5 nm.

Another preferred embodiment refers to a lyophilized formulation.

Another preferred embodiment refers to a lyophilized formulation (a lyophilizate), wherein the amount of a lipopeptide (b)), preferably a therapeutic lipopeptide, more preferably Bulevirtide or Liraglutide is between 12 mg and 24 mg, more preferably between 10 mg and 20 mg.

In yet another preferred embodiment refers to a lyophilized formulation (a lyophilizate), wherein the amount of a lipopeptide (b)), preferably a therapeutic lipopeptide, more preferably Liraglutide is between 0.5 mg and 1.8 mg such as 0.6 mg, 1.2 mg, or 1.8 mg, respectively.

In a lyophilized formulation, the sum of a), b), c), d) and e) sums up to 100% and the combined amount of a) to e) is between 98% and 100% based on the total weight of the formulation. Preferably, the combined amount of a) to e) is between 99% and 100%, even more preferably between 99.9% and 100%, most preferably 100%.

One preferred embodiment refers to a pharmaceutical liposomal formulation according to the invention comprises a), b), c), d) and e) and water and optionally a buffer.

Preferably, especially in the latter two preferred embodiments, the combined amount of a) to e) is between 10% and 15% based on the total weight of the formulation.

In a further preferred embodiment, the pharmaceutically active substance in the formulation according to the invention is entrapped by a lipid clathrate, proliposome, micelle or liposome, more preferably a liposome or a micelle. In other words, the composition comprises e.g., at least one liposome or at least one micelle, respectively, and Bulevirtide is loaded into the liposome or micelle.

In one preferred embodiment, a pharmaceutically active substance in the formulation according to the invention is entrapped by a micelle (a nano-dispersed formulation/a monophasic nano-disperse system). Most preferably the micelles have a D90 is 60 nm or less, such as less than 25 nm, more preferably 20 nm or less, such as between 10 nm and 20 nm, or between 3 nm and 10 nm.

In another preferred embodiment, a pharmaceutically active substance in the formulation according to the invention is entrapped by a liposome (a liposomal formulation).

Another preferred embodiment refers to a formulation wherein the formulation, preferably a liposomal formulation or a nano-dispersed formulation, is a pharmaceutical formulation.

### Liposomal Formulation

One preferred embodiment refers to a pharmaceutical liposomal formulation obtained by mixing/reconstituting the lyophilized formulation (monophasic nano-disperse system), preferably with an aqueous solution, more preferably with saline or water, even more preferably with saline.

Such a pharmaceutical liposomal formulation is preferably suitable for injection. In a particular embodiment, a liposomal formulation is intravenously or, preferably, subcutaneously administered to patients.

Another preferred embodiment refers to a pharmaceutical liposomal formulation wherein the lipopeptide is Bulevirtide and the amount of Bulevirtide in a liposomal formulation is between 12 mg/1.5 ml and 24 mg/1.5 ml, more preferably between 15 mg/1.5 ml and 20 mg/1.5 ml.

Another preferred embodiment refers to a pharmaceutical liposomal formulation wherein the lipopeptide is Liraglutide and the amount of Liraglutide in a liposomal formulation is between 0.5 mg/ml and 18 mg/ml such as between 0.5 mg/ml and 12 mg/ml, more preferably between 4 mg/ml to 8 mg/ml, even more preferably between 5.8 and 6.2 mg/ml such as 6 mg/ml.

According to another preferred embodiment, the pharmaceutical liposomal formulation has a pH between 5 and 7.5 such as between 5 and 6 or between 6 and 7.5 or between 7.3 and 7.4.

### a) Phospholipid

Another preferred embodiment refers to a formulation wherein the one or more phospholipid(s) comprises PC or a mixture of PC with one or more phospholipids selected from the group consisting of phosphatidylinositol (PI), phosphatidylserine (PS), phosphatidylethanolamine (PE), phosphatidylglycerol (PG) and phosphatidic acid (PA) phosphatidylglycerol (PG) or a pharmaceutically acceptable salt of any of the foregoing.

Another preferred embodiment refers to a formulation wherein the one or more phospholipid(s) is PC or a mixture of PC with one or more phospholipids selected from the group consisting of phosphatidylinositol (PI), phosphatidylserine (PS), phosphatidylethanolamine (PE), phosphatidylglycerol (PG) and phosphatidic acid (PA), phosphatidylglycerol (PG) or a pharmaceutically acceptable salt of any of the foregoing.

In one preferred embodiment, at least one phospholipid is a PEGylated phospholipid.

Another more preferred embodiment refers to a formulation wherein the one or more phospholipid(s) comprises PC or a, preferably pharmaceutically acceptable, salt thereof preferably selected from the group consisting of DLPC, DMPC, DPPC, DSPC, POPC, POPC, DEPC, HSPC, HEPC or a, preferably pharmaceutically acceptable, salt of any of the foregoing.

Another more preferred embodiment refers to a formulation wherein the one or more phospholipid(s) comprises a PEGylated PC or a, preferably pharmaceutically acceptable, salt thereof, preferably selected from the group consisting of a PEGylated DLPC, a PEGylated DMPC, a PEGylated DPPC, a PEGylated DSPC, a PEGylated POPC, a PEGylated POPC, a PEGylated DEPC, a PEGylated HSPC, a PEGylated HEPC or a, preferably pharmaceutically acceptable, salt of any of the foregoing.

Another more preferred embodiment refers to a formulation wherein the one or more phospholipid(s) comprises PC or a, preferably pharmaceutically acceptable, salt thereof. In one even more preferred embodiment, the PC or a, preferably pharmaceutically acceptable, salt thereof is from soybean (e.g. Lipoid S100), egg or synthetic, in a most preferred embodiment, the PC or a, preferably pharmaceutically acceptable, salt thereof is from soybean.

Another preferred embodiment refers to a formulation, wherein the amount of a) is between 50% and 65% compared to the total sum of a), b), c), d) and e) in the formulation. For example around 57%.

Another preferred embodiment refers to a formulation wherein PG or a, preferably pharmaceutically acceptable, salt thereof is a PEGylated PG or a PEGylated, preferably pharmaceutically acceptable, salt thereof.

Another more preferred embodiment refers to a formulation wherein the PG or a, preferably pharmaceutically acceptable, salt thereof is selected from the group consisting of DLPG, DMPG, DPPG, DSPG, POPG, POPG, DEPG, HSPG, HEPG.

Another more preferred embodiment refers to a formulation wherein the PG or a, preferably pharmaceutically acceptable, salt thereof is PEGylated and is selected from the group consisting of a PEGylated DLPG, a PEGylated DMPG, a PEGylated DPPG, a PEGylated DSPG, a PEGylated POPG, a PEGylated POPG, a PEGylated DEPG, a PEGylated HSPG, a PEGylated HEPG or a, preferably pharmaceutically acceptable, salt of any of the foregoing.

b) lipopeptide

In one preferred embodiment, a lipopeptide is a lipopeptide having not more than 50 amino acids.

In a further preferred embodiment, a lipopeptide has between 5 and 50 amino acids. Preferably, the between 5 and 50 amino acids are independently selected from the group consisting of alanine (ala), arginine (arg), asparagine (asn), aspartic acid (asp), cysteine (cys), glutamine (gln), glutamic acid (glu), glycine (gly), histidine (his), isoleucine (ile), leucine (leu), lysine (lys), methionine (met), phenylalanine (phe), proline (pro), serine (ser), threonine (thr), tryptophan (trp), tyrosine (tyr), and valine (val).

In another preferred embodiment, the lipophilic residue is attached to an amino acid via amidation, esterification (S— or O—) or S-bond (ether or disulphide) formation.

In another preferred embodiment, the lipopeptide has at least one lipophilic residue selected from the group consisting of glycosylphosphatidylinositol-anchor, palmitoyl (C16:0) or myristoyl (C14:0) residue covalently bond to one of the between 5 and 50 amino acids. E.g. a palmitoyl or myristoyl residue is attached to a cys residue.

In one preferred embodiment, a lipopeptide has one lipophilic residue selected from the group consisting of glycosylphosphatidylinositol-anchor, palmitoyl (C16:0) or myristoyl (C14:0) residue covalently bond to one of the between 5 and 50 amino acids.

In yet another preferred embodiment, a lipopeptide is one lipopeptide of the same kind (e.g., only Bulevirtide or only Liraglutide is present).

In another preferred embodiment, a lipopeptide used in a formulation (or method) according to the invention is Bulevirtide.

In yet another preferred embodiment, a lipopeptide used in a formulation (or method) according to the invention is Liraglutide.

In yet another preferred embodiment, the lipopeptide is Bulevirtide.

In yet another preferred embodiment, the lipopeptide is Liraglutide.

Another preferred embodiment refers to a formulation according to the invention, wherein Bulevirtide is used as its acetate salt. In one embodiment a formulation according to the invention comprises acetate, preferably in an equimolar range as Bulevirtide.

Another preferred embodiment refers to a formulation, wherein the amount of a lipopeptide (b)) is between 5% and 11% compared to the total sum of a), b), c), d) and e) in the formulation. For example around 8%.

In another preferred embodiment the amount of a lipopeptide, preferably Bulevirtide or Liraglutide is between 5% and 11% compared to the total sum of a), b), c), d) and e) in the formulation and the concentration of a lipopeptide (c)) in the formulation is between 15 mg/1.5 ml and 20 mg/1.5 ml, e.g., if the formulation is a liquid formulation such as monophasic nano-disperse system comprising Bulevirtide; or a liposomal formulation comprising a lipopeptide, preferably a pharmaceutical liposomal formulation.

In another preferred embodiment the amount of c) is between 5% and 11% compared to the total sum of a), b), c), d) and e) in the formulation and the concentration of c) in the formulation is between 8 mg/176.6 mg total sum of components a) to e) and 19.4 mg/176.6 mg total sum of components a) to e), e.g., if the formulation is a lyophilized formulation.

### c) Cholesterol

Cholesterol is known to have an influence on the stability of liposomes and on drug release.

Therefore, in one preferred embodiment, a formulation according to the invention preferably comprises cholesterol or a derivative thereof (component c)).

Another preferred embodiment refers to a formulation, wherein c) is selected from the group consisting of cholesterol or sodium cholesteryl sulfate. More preferably, component c) is cholesterol.

Another preferred embodiment refers to a formulation, wherein the amount of c) is between 6% and 12% compared to the total sum of a), b), c), d) and e) in the formulation. For example, around 9% such as between 8% and 10%.

### d) Bulking Agent

Especially when a formulation according to the invention should be lyophilizable or the preparation of a formulation according to the invention via a method according to the invention requires a lyophilization step, the presence of a bulking agent is advantageous. Examples of preferred bulking agents are glycine, arginine, proline, or any other amino acid known to be suitable as a bulking agent or a saccharide component (component e) in a formulation according to the invention). Preferred saccharide components are selected from the group consisting of sucrose, trehalose, arabinose, erythritol, fructose, galactose, glucose, lactose, maltitol, maltose, maltotriose, mannitol, mannobiose, mannose, ribose, sorbitol, xylitol, xylose, dextran, or a mixture thereof.

One more preferred embodiment refers to a formulation, wherein e) is trehalose.

Another preferred embodiment refers to a formulation, wherein the amount of e) is between 20% and 30% compared to the total sum of a), b), c), d) and e) in the formulation. For example around 25%.

In one preferred embodiment, a formulation according to the invention comprises in addition to components a) to e) tert-butanol in a range between 0.01% and 2% compared to the total weight of the formulation (e.g. a lyophilizate).

### e) Tonicity Adjusting Agents

Another preferred embodiment refers to a pharmaceutical liposomal formulation comprising a), b), c), d) and one or more tonicity adjusting agent(s) which is not d) (component e) in a formulation according to the invention).

Such a tonicity adjusting agent, preferably a pharmaceutically acceptable tonicity adjusting agent, should be present in a pharmaceutical liposomal formulation. It can be added during preparing the liposomal formulation by mixing a lyophilizate according to the invention with an aqueous phase comprising said tonicity adjusting agent or it can be provided during a rehydration step of a lyophilizate.

Alternatively, it can already be present in the lyophilizate in that the tonicity adjusting agent is part of the aqueous phase which is used in a method according to the invention to prepare a lipopeptide according to the invention. The skilled person is aware that a tonicity agent can also be partially be part of such an aqueous phase and partially part of an aqueous phase. The skilled person can easily calculate the total amount of a tonicity adjusting agent which is required to have the right concentration in the final pharmaceutical liposomal formulation to have an isotonic effect in regard of a medicament which is provided to a patient.

The skilled person will recognize that a tonicity adjusting agent is in some cases also a bulking agent, e.g. in case the tonicity agent is a saccharide component such as glucose or trehalose.

Other tonicity adjusting agents such as NaCl do not have a bulking function. Thus, if using such a “single functional component”, e.g., NaCl, a formulation may comprise a bulking agent e) and a tonicity adjusting agent e). It may even comprise a bulking agent (e.g. trehalose) as well as “two” tonicity adjusting agents (trehalose and, e.g., NaCl).

Tonicity adjusting agents are well known to the skilled person. In one preferred embodiment, the tonicity adjusting agent is selected from the group consisting of dextrose, glucose, mannitol, sucrose, lactose, trehalose, glycine, arginine, proline and NaCl, in particular glycine, mannitol, trehalose, glucose and NaCl, even more particularly glycine and NaCl, most preferably NaCl.

The skilled person is aware how to choose the right concentration of a specific tonicity adjusting agent in a formulation, preferably for subcutaneous injection.

In a preferred embodiment, the tonicity adjusting agents is NaCl and the concentration of NaCl in the pharmaceutical liposomal formulation is between 0.8% and 1%, more preferably around 0.9%, e.g. 0.9%+0.1%, more preferably 0.9%, based on the total weight of the pharmaceutical liposomal formulation.

In one preferred embodiment, component e) is present in a formulation according to the invention in an amount of between 0.1% and 10%, more preferably between 0.4% and 1.5%, for example, when the tonicity agent is NaCl, the preferred amount of NaCl in a formulation such as a pharmaceutically acceptable formulation is between 0.8% and 1.0% (0.9%+0,1%, more preferably 0.9%+0.05%), i.e. in regard of blood plasma, in a physiological concentration or close to a physiological concentration.

Depending on the tonicity adjusting agent and the kind of the formulation (e.g. a pharmaceutically acceptable liposomal formulation or a lyophilizate), the skilled person is well aware how to calculate the amount of one or more tonicity agent(s) present in a (final) formulation for administration to a patient with a physiological concentration.

### Solvent for Aqueous Solution

Another preferred embodiment refers to a pharmaceutical liposomal formulation comprising a), b), c), d) and e) as defined herein and one or more solvent(s).

Another preferred embodiment refers to a pharmaceutical liposomal formulation wherein, the one or more solvents is present in an amount between 10% and 90% based on the total weight of the final formulation, such as between 80% and 90% or even between 85% and 90%.

Appropriate solvents can be selected by the skilled formulator. In one preferred embodiment, the solvent or solvents are selected from the group consisting of water, and an aqueous solution in which for example salts are present. An aqueous solution in which salts are present is preferably a solution, preferably water, comprising a physiological concentration of at least one salt, preferably an isotonic concentration of at least one salt, like for example saline, Ringer's lactate solution and/or Plasma Lyte. Herein, saline can also be referred to as saline solution and relates to a mixture of sodium chloride and water, preferably with a sodium chloride concentration of 9 g of salt per litre (0.9%) solution (see also Example 4, in which such 0.9% (w/v) saline is used for reconstitution and formation of liposomes). Ringer's lactate solution refers to a sodium lactate solution as a mixture of sodium chloride, sodium lactate, potassium chloride, and calcium chloride in water. Plasma Lyte may also be referred to as Plasma-lyte 148 (pH 7.4) Thus, said aqueous solution has preferably a salt concentration, osmolality and pH that reflects a human physiological plasma electrolyte concentrations, osmolality and pH. However, in a more preferred embodiment, the solvent is water or a combination of water with, preferably an isotonic concentration of, at least one salt, wherein the amount of water of the total amount of solvent is at least 80%, preferably at least 90%, more preferably at least 95%. Preferably the solvent is water, saline, Ringer's lactate solution or Plasma Lyte.

More preferably, the one or more solvent is water or saline. Most preferably, the one or more solvent is water.

### Buffer

Another preferred embodiment refers to a formulation, wherein the formulation is a pharmaceutical liposomal formulation comprising a), b), c), d) and e) as defined herein and a buffer.

Another preferred embodiment refers to a formulation, wherein the formulation is a pharmaceutical liposomal formulation consisting of a), b), c), d) and e) as defined herein, and a solvent.

Another preferred embodiment refers to a formulation, wherein the formulation is a pharmaceutical liposomal formulation comprising a), b), c), d) and e) as defined herein, a solvent and one or more tonicity adjusting agent(s) and a buffer.

Any pharmaceutically acceptable components in addition to components a) to e) in a pharmaceutical liposomal formulation according to the invention such as one or more solvent(s), buffer(s), salts, other additives are summarized as pharmaceutical aqueous solution. Thus, a pharmaceutical liposomal formulation according to the invention consists of components a) to e) and the pharmaceutical aqueous solution. The skilled person will understand that components forming the pharmaceutical aqueous solution can be sequentially combined with a formulation comprising a) to e) as defined herein or can be first mixed and then added to a formulation comprising a) to e) as defined herein (i.e. this formulation may only comprise a) to e) and, e.g., a buffer or is provided together with/in a pharmaceutical aqueous solution or f) and the aqueous solution are provided sequentially, resulting in a pharmaceutical liposomal formulation consisting of components a) to e) and a pharmaceutical aqueous solution according to the invention.

### Vesicle Size of the Monophasic Nano-Disperse System

In yet another embodiment, the lipopeptide formulation of step iv) of a method according to the invention (see further below) is a monophasic nano-dispersed system, wherein the D90 of the vesicles is 60 nm or less, more preferably less than 25 nm, even more preferably 20 nm or less such as between 10 nm and 20 nm, or between 3 nm and 10 nm.

### Liposome Size

In yet another embodiment, the liposomes of a liposomal formulation according to the invention have a D90 size distribution of between 1 μm and 4.5 μm.

### Method of Treatment/Use

One aspect refers to the use of a formulation according to the invention wherein the lipopeptide is Bulevirtide for the preparation of a medicament to treat chronic hepatitis B and/or chronic hepatitis D.

Another aspect refers to the use of a formulation according to the invention wherein the lipopeptide is Bulevirtide for the preparation of a medicament to treat inflammatory, preferably an inflammatory disease.

Another aspect refers to a method for treating chronic hepatitis B and/or chronic hepatitis D comprising the step of providing to a patient a pharmaceutical liposomal formulation according to the invention wherein the lipopeptide is Bulevirtide. Preferably, the provision of the pharmaceutical liposomal formulation according to the invention is an injection.

One aspect refers to the use of a formulation according to the invention wherein the lipopeptide is Liraglutide for the preparation of a medicament to treat type 2 diabetes.

Another aspect refers to a method for treating type 2 diabetes comprising the step of providing to a patient a pharmaceutical liposomal formulation according to the invention wherein the lipopeptide is Liraglutide. Preferably, the provision of the pharmaceutical liposomal formulation according to the invention is an injection.

### Method of Preparation

Another aspect refers to a method of preparing a formulation according to the invention comprising the steps of:

i) providing an organic phase comprising one or more phospholipid(s) (component a) in a formulation according to the invention), optionally Cholesterol or a derivative thereof (component c) in a formulation according to the invention), and at least one organic solvent, preferably selected from the group consisting anisole, ethylacetate, 1,4-dioxane, dimethylcarbonate, dimethylsulfoxide, glycofurol, N, N-dimethylacetamide, N,N-dimethylformamide, N-methyl-2-pyrrolidone (NMP), isopropylideneglycerol, an alcohol, preferably an alcohol selected from the group consisting of 1-butanol, 2-butanol and tert-butanol, more preferably tert-butanol, acetic acid, ethyl lactate (ethyl 2-hydroxypropanoate), acetonitrile, and a combination of any of the foregoing;
ii) providing an aqueous phase comprising an aqueous medium; and
   optionally a bulking agent selected from the group consisting of glycine, arginine, proline or any other amino acid known to be suitable as a bulking agent, a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture of any of the foregoing,
   optionally a pharmaceutically acceptable buffer, and
   optionally a pharmaceutically acceptable tonicity adjusting agent,
   wherein the pH of the aqueous phase is between 3 and 9, e.g., between 5.5 and 7, such as between 5.8 and 6.7 (for example, by using a sodium acetate buffer);
iii) Combining the organic phase and the aqueous phase, wherein the mixing ratio of the organic phase and the aqueous phase is between 10:1 (v/v) and 1:10 (v/v) resulting in a combined organic and aqueous phase, wherein said at least one organic solvent and the aqueous phase form a monophasic mixture, preferably a freezable and sublimable monophasic mixture;
iv) adding a lipopeptide, preferably Bulevirtide or Liraglutide, more preferably Bulevirtide, to the combined phases to receive a lipopeptide formulation according to the invention; or

adding a lipopeptide, preferably Bulevirtide or Liraglutide, more preferably Bulevirtide, to the organic phase before mixing the organic phase with the aqueous phase in step iii); or

adding a lipopeptide, preferably Bulevirtide or Liraglutide, more preferably Bulevirtide, to the aqueous phase before mixing the aqueous phase with the organic phase in step iii)

resulting in a lipopeptide formulation, wherein said lipopeptide formulation so obtained is a monophasic nano-disperse system.

Preferably, the aqueous phase comprises a buffer.

One preferred embodiment refers to a method of preparing a formulation according to the invention comprising the steps of:
i) providing an organic phase comprising one or more phospholipid(s) (component a) in a formulation according to the invention), optionally Cholesterol or a derivative thereof (component c) in a formulation according to the invention), and at least one organic solvent, preferably selected from the group consisting anisole, ethylacetate, 1,4-dioxane, dimethylcarbonate, dimethylsulfoxide, glycofurol, N,N-dimethylacetamide, N,N-dimethylformamide, N-methyl-2-pyrrolidone (NMP), isopropylideneglycerol, an alcohol, preferably an alcohol selected from the group consisting of 1-butanol, 2-butanol and tert-butanol, more preferably tert-butanol, acetic acid, ethyl lactate (ethyl 2-hydroxypropanoate), acetonitrile, and a combination of any of the foregoing;
ii) providing an aqueous phase comprising an aqueous medium; and
   optionally a bulking agent selected from the group consisting of glycine, arginine, proline or any other amino acid known to be suitable as a bulking agent, a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture of any of the foregoing,
   a pharmaceutically acceptable buffer, and
   optionally a pharmaceutically acceptable tonicity adjusting agent,
wherein the pH of the aqueous phase is between 3 and 9, e.g., between 5.5 and 7, such as between 5.8 and 6.7 (for example, by using a sodium acetate buffer);
iii) Combining the organic phase and the aqueous phase, wherein the mixing ratio of the organic phase and the aqueous phase is between 10:1 (v/v) and 1:10 (v/v) resulting in a combined organic and aqueous phase, wherein said at least one organic solvent and the aqueous phase form a monophasic mixture, preferably a freezable and sublimable monophasic mixture;
iv) adding a lipopeptide, preferably Bulevirtide or Liraglutide, more preferably Bulevirtide, to the combined phases to receive a lipopeptide formulation according to the invention; or adding a lipopeptide, preferably Bulevirtide or Liraglutide, more preferably Bulevirtide, to the organic phase before mixing the organic phase with the aqueous phase in step iii); or

adding a lipopeptide, preferably Bulevirtide or Liraglutide, more preferably Bulevirtide, to the aqueous phase before mixing the aqueous phase with the organic phase in step iii)

resulting in a lipopeptide formulation, wherein said lipopeptide formulation so obtained is a monophasic nano-disperse system.

Thus, another preferred embodiment refers to a method of preparing a formulation according to the invention comprising the steps of:
i) providing an organic phase comprising one or more phospholipid(s) (component a) in a formulation according to the invention), optionally Cholesterol or a derivative thereof (component c) in a formulation according to the invention), and at least one organic solvent, preferably selected from the group consisting anisole, ethylacetate, 1,4-dioxane, dimethylcarbonate, dimethylsulfoxide, glycofurol, N, N-dimethylacetamide, N,N-dimethylformamide, N-methyl-2-pyrrolidone (NMP), isopropylideneglycerol, an alcohol, preferably an alcohol selected from the group consisting of 1-butanol, 2-butanol and tert-butanol, more preferably tert-butanol, acetic acid, ethyl lactate (ethyl 2-hydroxypropanoate), acetonitrile, and a combination of any of the foregoing;
ii) providing an aqueous phase comprising an aqueous medium; and
   optionally a bulking agent selected from the group consisting of glycine, arginine, proline or any other amino acid known to be suitable as a bulking agent, a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture of any of the foregoing,
   optionally a pharmaceutically acceptable buffer, and
   optionally a pharmaceutically acceptable tonicity adjusting agent,
   wherein the pH of the aqueous phase is between 3 and 9, e.g., between 5.5 and 7, such as between 5.8 and 6.7 (for example, by using a sodium acetate buffer);
iii) Combining the organic phase and the aqueous phase, wherein the mixing ratio of the organic phase and the aqueous phase is between 10:1 (v/v) and 1:10 (v/v) resulting in a combined organic and aqueous phase, wherein said at least one organic solvent and the aqueous phase form a monophasic mixture, preferably a freezable and sublimable monophasic mixture;
iv) adding a lipopeptide, preferably Bulevirtide or Liraglutide, more preferably Bulevirtide, to the combined phases to receive a lipopeptide formulation according to the invention, wherein said lipopeptide formulation so obtained is a monophasic nano-disperse system.

Preferably, the aqueous phase comprises a buffer.

Another embodiment refers to a method of preparing a formulation according to the invention comprising the steps of:
i) providing an organic phase comprising one or more phospholipid(s) (component a) in a formulation according to the invention), a lipopeptide, preferably Bulevirtide or Liraglutide, more preferably Bulevirtide (component b) in a formulation according to the invention), optionally Cholesterol or a derivative thereof (component c) in a formulation according to the invention), and at least one organic solvent, preferably selected from the group consisting anisole, ethylacetate, 1,4-dioxane, dimethylcarbonate, dimethylsulfoxide, glycofurol, N,N-dimethylacetamide, N,N-dimethylformamide, N-methyl-2-pyrrolidone (NMP), isopropylideneglycerol, an alcohol, preferably an alcohol selected from the group consisting of 1-butanol, 2-butanol and tert-butanol, more preferably tert-butanol, acetic acid, ethyl lactate (ethyl 2-hydroxypropanoate), acetonitrile, and a combination of any of the foregoing;
ii) providing an aqueous phase comprising an aqueous medium; and
   optionally a bulking agent selected from the group consisting of glycine, arginine, proline or any other amino acid known to be suitable as a bulking agent, a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture of any of the foregoing,
   optionally a pharmaceutically acceptable buffer, and
   optionally a pharmaceutically acceptable tonicity adjusting agent,
wherein the pH of the aqueous phase is between 3 and 9, e.g., between 5.5 and 7, such as between 5.8 and 6.7 (for example, by using a sodium acetate buffer);
iii) Combining the organic phase and the aqueous phase, wherein the mixing ratio of the organic phase and the aqueous phase is between 10:1 (v/v) and 1:10 (v/v) resulting in a combined organic and aqueous phase, wherein said at least one organic solvent and the aqueous phase form a monophasic mixture, preferably a freezable and sublimable monophasic mixture.

Preferably, the aqueous phase comprises a buffer.

Another embodiment refers to a method of preparing a formulation according to the invention comprising the steps of:
i) providing an organic phase comprising one or more phospholipid(s) (component a) in a formulation according to the invention), optionally Cholesterol or a derivative thereof (component c) in a formulation according to the invention), and at least one organic solvent, preferably selected from the group consisting anisole, ethylacetate, 1,4-dioxane, dimethylcarbonate, dimethylsulfoxide, glycofurol, N, N-dimethylacetamide, N,N-dimethylformamide, N-methyl-2-pyrrolidone (NMP), isopropylideneglycerol, an alcohol, preferably an alcohol selected from the group consisting of 1-butanol, 2-butanol and tert-butanol, more preferably tert-butanol, acetic acid, ethyl lactate (ethyl 2-hydroxypropanoate), acetonitrile, and a combination of any of the foregoing;
ii) providing an aqueous phase comprising an aqueous medium; and
   a lipopeptide, preferably Bulevirtide or Liraglutide, more preferably Bulevirtide (component b) in a formulation according to the invention),
   optionally a bulking agent selected from the group consisting of glycine, arginine, proline or any other amino acid known to be suitable as a bulking agent, a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture of any of the foregoing,
   optionally a pharmaceutically acceptable buffer, and
   optionally a pharmaceutically acceptable tonicity adjusting agent,
wherein the pH of the aqueous phase is between 3 and 9, e.g., between 5.5 and 7, such as between 5.8 and 6.7 (for example, by using a sodium acetate buffer), and
iii) Combining the organic phase and the aqueous phase, wherein the mixing ratio of the organic phase and the aqueous phase is between 10:1 (v/v) and 1:10 (v/v) resulting in a combined organic and aqueous phase, wherein said at least one organic solvent and the aqueous phase form a monophasic mixture, preferably a freezable and sublimable monophasic mixture.

Preferably, the aqueous phase comprises a buffer.

For explanation sake, a mixing ratio of 10:1 (v/v) means 10 volume parts of an organic phase and 1 volume part of an aqueous phase are combined (e.g. 10 ml and 1 ml). Preferably, the mixing ratio in a method according to the invention is between 5:1 (v/v) and 1:5 (v/v) such as 1:1 or around 1, e.g. 2:1 to 1:2.

### Aqueous Medium

The aqueous medium is preferably water or an aqueous solution in which for example salts are present. An aqueous solution in which salts are present is preferably a solution, preferably water, comprising a physiological concentration of at least one salt, preferably an isotonic concentration of at least one salt, like for example saline, Ringer's lactate solution and/or Plasma Lyte. Thus, the aqueous medium has preferably a salt concentration, osmolality and pH that reflects a human physiological plasma electrolyte concentrations, osmolality and pH. The aqueous medium is preferably water or a combination of water with, preferably an isotonic concentration of, at least one salt, preferably saline, Ringer's lactate solution and/or Plasma Lyte The amount of water of the total amount of solvent of the aqueous medium is at least 80%, preferably at least 90%, more preferably at least 95%. More preferably, the aqueous medium is water or saline. Most preferably, the aqueous medium is water.

Preferred buffers, bulking agents and tonicity adjusting agents for use in a method according to the invention are identical to the respective buffers, bulking agents and tonicity adjusting agents described for the formulations of the invention.

### Aqueous Phase

The aqueous phase in step ii) comprises an aqueous medium, preferably the aqueous medium consists of water, i.e. water is the only solvent in an aqueous phase.

In one preferred embodiment, the pH of an aqueous phase is chosen so that the lipopeptide is substantially not soluble in the aqueous phase. Substantially not soluble in this regard means that at most 20% (w/w) (compared to the total amount mixed with an aqueous phase in ii)), more preferably at most 10% (w/w) even more preferably at most 5% (w/w), even more preferably at most 1% (w/w), most preferably at most 0.1% (w/w) of a lipopeptide is soluble in the aqueous phase.

Preferred buffers, bulking agents and tonicity adjusting agents for use in a method according to the invention are identical to the respective buffers, bulking agents and tonicity adjusting agents described for the formulations of the invention.

### Buffer for Aqueous Phase (Step i))

In one preferred embodiment, an aqueous phase comprises a buffer.

The skilled person is aware how to choose a buffer for a specific pH value. Preferred buffers are acetate buffer (e.g., acetate/acetic acid) (preferably for pH between 3.7 to 6.5)), phosphate buffer or citrate buffer (e.g., Na₂HPO₄/citric acid, Na₂HPO₄/NaH₂PO₄ or Na₂HPO₄/NaOH) (preferably for pH between 5.4 and 8.0), sodium citrate/citric acid (preferred pH between 3.0 and 6.2)).

In one preferred embodiment, the aqueous phase comprises at least 70% (w/w), more preferably at least 90% (w/w) aqueous medium, preferably water, optionally a bulking agent (component d) in a formulation according to the invention), wherein the amount of the bulking agent in the aqueous phase is preferably between 1% (w/w) and 25% (w/w), and a buffer, wherein the amount of buffer is preferably between 0.001% (w/w) and 5% (w/w), more preferably between 0.001% (w/w) and 1% (w/w) such as between 0.01% (w/w) and 0.1% (w/w).

In case a bulking agent is present in an aqueous phase, the skilled person is aware how to choose the total volume of an aqueous phase and the amount of a bulking agent (and the amount of further optional components such as a buffer or a tonicity agent different from the bulking agent) to arrive at a formulation according to the invention.

In another preferred embodiment, water is the only solvent in an aqueous medium and, thus, an aqueous phase.

Most preferred are acetate buffers, especially when Bulevirtide is the lipopeptide b). Preferably, the pH of such an aqueous phase is between 5 and 6.8, such as between 5 and 6.

In a further preferred embodiment, the aqueous phase consists of an aqueous medium, a bulk agent, a buffer, and optionally a lipopeptide. If a lipopeptide is present in an aqueous phase, the pH of said aqueous phase should be in a range in which a lipopeptide is soluble in the aqueous phase. In case a lipopeptide is not soluble in an aqueous phase, it is advantageous to add the lipopeptide to the organic phase or to the combined organic and aqueous phase.

In yet a further preferred embodiment, the aqueous phase consists of water, a bulk agent, a buffer; and optionally a lipopeptide.

In yet a further preferred embodiment, the aqueous phase consists of an aqueous medium, preferably water, a buffer and optionally a lipopeptide.

In a very preferred embodiment, all components such as an aqueous medium, a buffer and a tonicity adjusting agent are pharmaceutically acceptable components.

Usually, a buffer is present in a concentration between 0.05 mM and 100 mM such as between 1 mM and 50 mM.

### Organic Phase

Besides definitions and illustrative examples as regards the at least one organic solvent given herein, for the organic phase, at least one appropriate water-miscible lyophilizable organic solvent can be selected by the skilled formulator.

The organic solvent should be miscible with water at standard conditions (25° C. and 1.013 bar). Moreover, an organic solvent should be lyophilizable so that the organic solvent can be removed by sublimation (the transition of a substance directly from the solid to the gas state, without passing through the liquid state). The skilled person is aware how to choose suitable organic solvents as well as suitable temperatures and pressures to lyophilize an organic solvent by, e.g., using a pressure temperature (PT) diagram of an organic solvent known in the art.

Preferred organic solvents are selected from the group consisting of anisole, ethylacetate, 1,4-dioxane, dimethylcarbonate, dimethylsulfoxide, glycofurol, N,N-dimethylacetamide, N,N-dimethylformamide, N-methyl-2-pyrrolidone (NMP), isopropylideneglycerol and alcohols, like butanol, preferably selected from the group consisting of 1-butanol, 2-butanol and tert-butanol, acetic acid, ethyl lactate (ethyl 2-hydroxypropanoate), acetonitrile, or a combination of any of the foregoing, preferably the at least one organic solvent is selected from the group consisting of an alcohol, preferably tert-butanol, anisole, dimethylsulfoxide, 1,4-dioxane and dimethylcarbonate and a combination thereof.

In one preferred embodiment, the organic solvent is anisole (CAS 100-66-3).

In one preferred embodiment, the organic solvent is ethylacetate (CAS 141-78-6).

In one preferred embodiment, the organic solvent is 1,4-dioxane (CAS 123-91-1).

In one preferred embodiment, the organic solvent is dimethylcarbonate (CAS 616-38-6).

In one preferred embodiment, the organic solvent is dimethylsulfoxide (CAS 67-68-5).

In one preferred embodiment, the organic solvent is glycofurol (CAS 31692-85-0).

In one preferred embodiment, the organic solvent is N,N-dimethylacetamide (CAS 127-19-5).

In one preferred embodiment, the organic solvent is N,N-dimethylformamide (CAS 68-12-2).

In one preferred embodiment, the organic solvent is N-methyl-2-pyrrolidone (CAS 872-50-4).

In one preferred embodiment, the organic solvent is isopropylideneglycerol (CAS 100-79-8).

In one preferred embodiment, the organic solvent is 1-butanol (CAS 71-36-3).

In one preferred embodiment, the organic solvent is 2-butanol (CAS 78-92-2).

In one preferred embodiment, the organic solvent is tert-butanol (CAS 75-65-0).

In one preferred embodiment, the organic solvent is acetic acid.

In one preferred embodiment, the organic solvent is ethyl lactate (ethyl 2-hydroxypropanoate).

In one preferred embodiment, the organic solvent is acetonitrile.

It was surprisingly found that the use of butanol, preferably tert-butanol (TBA) leads to a preferred monophasic nano-disperse system when combining the organic phase from step i) with the aqueous phase from step ii).

Thus, in one more preferred embodiment, the organic solvent is tert-butanol (CAS 75-65-0).

Although higher as well as lower concentrations of a lipopeptide component in an organic phase suitable for a method according to the invention can be used, it is preferred the sum of the one or more lipopeptide(s) in an organic phase is between 3% and 30%, e.g. between 5% and 25% such as between 10% and 20%, based on the total weight of the organic phase.

The skilled person is able to calculate the concentrations and amounts of the various components for the organic phase and aqueous phase to be combined in step iii) to arrive at a formulation according to the invention with the requirements regarding the ratios between the various components a) to e), without undue burden.

Surprisingly, it was found the lipopeptide formulation of step iv) is a monophasic nano-dispersed system, wherein the D90 of the vesicles is 60 nm or less, preferably less than 25 nm, more preferably 20 nm or less, such as between 10 nm and 20 nm e.g., around 15 nm, or between 3 nm and 10 nm, e.g., around 5 nm.

In other words, being bound to the explanation, by combining the organic phase from i) and the aqueous phase from ii) no liposomes are resulting but a monophasic nano-disperse system with a D90 (of the particle) size of the micelles of 60 nm or less, preferably less than 25 nm. This nano-dispersion can be sterile filtered.

In one preferred embodiment, the ratio between the organic phase and the aqueous phase is between 2:1 (v/v) and 1:4,5(v/v), even more preferably between 1,5:1 (v/v) and 1:4 (v/v) such as between 1:1 (v/v) to 1:4 (v/v), e.g. between 1:1 (v/v) and 1:3.5 (v/v), such as around 1:1 (v/v), around 1:2 (v/v) or around 1:3 (v/v). For explanation sake, “(v/v)” refers to volume ratios of the two phases, e.g., a ratio of 1:1 refers to 1 ml of an organic phase and 1 ml of an aqueous phase.

In one preferred embodiment, the D90 of the vesicles in a resulting monophasic nano-dispersed system, when the ratio between the two phases is 1:1, is 15 nm or less, more preferably 10 nm or less such as between 3 nm and 10 nm. Without being bound to the explanation, the high ratio of organic solvent may lead to a predominantly molecular solution of the components within the solvent mixture.

In another preferred embodiment, the D90 of the vesicles in a monophasic nano-dispersed system, when the ratio between the two phases is 1:3, is 60 nm or less, more preferably less than 25 nm, even more preferably 20 nm or less such as between 5 nm and 20 nm. Without being bound to the explanation, at this higher proportion of water a micellar solution is formed showing the typical size and homogeneity of micelles. The mixture shows a nearly clear appearance with slight Tyndall effect and is freely filterable through a sterile filter with 0.22 μm nominal pore size.

At a solvent mixture ratio of 1:5 the resulting preparation is turbid. Size distribution measurement by DLS reveals a broad inhomogeneous spectrum of vesicles having a mean size of about 1,000 μm (1000 nm). This dispersion can be filtered through a membrane sterile filter with 0.22 μm nominal pore size only by applying high pressure.

In one preferred embodiment, the ratio between the organic phase is between 1:1 and 1:4 and the size of the vesicles, preferably micelles, in the monophasic nano-dispersed system is between 5 nm and 60 nm.

The methods according to the invention allow a high loading of a lipopeptide into vesicles. After lyophilization, small portion of the organic solvent, preferably butanol, more preferably tert-butanol, may still be present in the lyophilizate. Thus, a formulation according to the invention may still comprise small amounts of the organic solvent, preferably butanol, more preferably tert-butanol, when prepared according to a method of the invention.

The skilled person will understand that the order of step i) and step ii) is exchangeable. Likewise, the skilled person will understand that PG and the phospholipid a) can each be dissolved in an amount of an organic solvent, separately, and then the two organic phases can be combined to result in the organic phase of step i) or both compounds can be in parallel or sequentially dissolved in the same amount of an organic solvent resulting in the organic phase of step i).

Preferred lipopeptides for the method according to the invention are also described for the formulations according to the invention, above.

Usually, step i) to iv) can be performed around and at room temperature (25° C.) and around and at standard pressure (101.325 kPa). Generally, steps i), ii), iii), and iv) can individually be performed at temperatures preferably between 0° C. and 40° C., more preferably between 15° C. and 35° C. such as between 18° C. and 28° C. Although any of the four steps can be individually carried out also at higher and lower pressures, preferably any of the steps is individually carried out at a pressure between 90 kPa and 112 kPa, more preferably between 95 kPa and 116 kPa, most preferably around standard pressure, e.g., 101.325 kPa+2%.

Optionally, the method comprises one or more further steps.

Preferably one further step is the step “sterile filtering”:
sterile filtering the resulting formulation comprising a), b), d), and optionally e), and optionally d).

Preferably, the sterile filtering is done by using a membrane filter, e.g. a PVDF membrane filter. Preferably, the nominal pore size of a membrane filter is 200 nm or less.

The same preferred temperatures and pressures as for the steps i) to iv) also apply to a sterile filtering step.

Optionally, the method comprises further the optional step “lyophilization” of the nano-dispersed formulation with the lipopeptide:
lyophilizing the formulation resulting from step iv) (or a sterile filtering step after step iv) comprising a), b), c), d) and e) as defied herein resulting in a lyophilizate comprising a), b), c), d) and e) as defined herein.

The skilled person is well-aware of freeze drying (lyophilization) techniques. Usually, freeze drying of a formulation according to the invention is performed at a temperature between +40° C. and −40° C., preferably between +30° C. and −10° C. A freeze-drying step may be repeated one or more times. Usually, the pressure is between 1000 hPa and 0.001 hPa (1 hPa=1 mbar). Preferably, a freeze dying step is performed at a pressure between 1 hPa and 0.01 hPa such as around 0.1 hPa.

Optionally, the method comprises a step of lyophilization and the method further comprises a step of rehydrating a lyophilizate comprising a), b), d), and optionally c), and optionally e) with a, preferably pharmaceutical, aqueous solution.

Another preferred embodiment, refers to a method of preparing a formulation according to the invention comprising the steps of:
i) preparing an organic phase by dissolving a phospholipid (a)), preferably comprising PC and PG (component a)) and, and optionally Cholesterol (component c)) in butanol, preferably tert-butanol;
ii) preparing an aqueous phase by dissolving a saccharide component d), preferably trehalose, and optionally a tonicity adjusting agent which is not component d) (component e)) in an aqueous medium preferably wherein the aqueous medium has a pH between 3 and 9, e.g., around 5.5 (for example, by using a sodium acetate buffer);
iii) combining the organic phase and the aqueous phase;
iv) adding a lipopeptide, preferably Bulevirtide or Liraglutide, more preferably Bulevirtide either to the organic phase of step i) and then mixing the organic phase with the lipopeptide with the aqueous phase as described in step iii), or to the aqueous phase of step ii) and then mixing the aqueous phase with the lipopeptide with the organic phase as described in step iii), or to the combined phases of step iii), preferably to the combined phases of step iii), resulting in a lipopeptide formulation, wherein said lipopeptide formulation so obtained is a monophasic nano-disperse system;
v) lyophilizing the formulation of step iv) resulting in a lyophilizate comprising a), b), d), and optionally c), and optionally e).

Optionally, the method comprises further steps such as a step “sterile filtering” step, preferably before step iv) or before step v).

Another preferred embodiment refers to a method for the preparation of a liposomal formulation, preferably a pharmaceutical liposomal formulation according to the invention comprising the steps of
i) preparing an organic phase by dissolving a phospholipid (a)), preferably comprising PC and PG, (component a)) and, and optionally Cholesterol (component c) in butanol, preferably tert-butanol;
ii) preparing an aqueous phase by dissolving a saccharide component e), preferably trehalose, in an aqueous medium preferably wherein the aqueous medium has a pH between 3 and 9, e.g., around 5,5 (for example, by using a sodium acetate buffer);
iii) Combining the organic phase and the aqueous phase;
iv) adding a lipopeptide, preferably Bulevirtide or Liraglutide, more preferably Bulevirtide, to the combined phases to receive a lipopeptide formulation according to the invention; or adding a lipopeptide, preferably Bulevirtide or Liraglutide, more preferably Bulevirtide, to the organic phase before mixing the organic phase with the aqueous phase in step iii); or adding a lipopeptide, preferably Bulevirtide or Liraglutide, more preferably Bulevirtide, to the aqueous phase before mixing the aqueous phase with the organic phase in step iii).
v) lyophilizing the lipopeptide formulation of step iv) resulting in a lyophilizate comprising a), b), d), and optionally c) and optionally e).
vi) rehydrating the lyophilizate of step v) with a, preferably pharmaceutical, aqueous solution resulting in a, preferably pharmaceutical, liposomal formulation.

Another preferred embodiment, refers to a method for the preparation of a pharmaceutical liposomal formulation according to the invention comprising a step of “rehydration” of a lyophilizate comprising:

rehydrating a lyophilizate comprising a), b), c), optionally d), e) and optionally f), preferably prepared according to the steps i) to iv) as described above and at least the optional step “lyophilization” outlined above with a pharmaceutical aqueous solution.

Usually, such a step can be performed at the same temperatures and pressures as described for steps i) to iv).

For homogenization purpose, it is sufficient to gently swirl or vortex the obtained liposomal dispersion. It is not required to reduce the size of the liposomes by further, complicated steps, when the liposomal formulation should be suitable for injection into a patient in need of a lipopeptide formulation.

In one preferred embodiment, the ratio between a lipopeptide and the one or more phospholipid(s) in an organic phase, a combined organic and aqueous phase, a lipopeptide formulation, a lyophilizate or a liposomal formulation, respectively, is between 1:333 and 1:2 such as between 1:333 and 1:49 (if the lipopeptide is, e.g., Liraglutide) or between 1:33 and 1:6.7 (if the lipopeptide is, e.g., Bulevirtide) based on the total amount of lipopeptide and phospholipid(s) in said organic phase, combined organic and aqueous phase, lipopeptide formulation, lyophilizate or liposomal formulation, respectively.

### Aqueous Solution

A, preferably pharmaceutical, aqueous solution as used herein comprises a pharmaceutical aqueous medium, preferably water or an (preferably pharmaceutical) aqueous medium in which for example salts are present. Said aqueous medium in which salts are present is preferably an aqueous medium, preferably water, comprising a physiological concentration of at least one salt, preferably an isotonic concentration of at least one salt, like for example saline, Ringer's lactate solution and/or Plasma Lyte. Thus, the (preferably pharmaceutical) aqueous medium has preferably a salt concentration, osmolality and pH that reflects a human physiological plasma electrolyte concentrations, osmolality and pH. Preferably, the, preferably pharmaceutical, aqueous solution is water or a combination of water with, preferably an isotonic concentration of, at least one salt, wherein preferably wherein the amount of water of the total amount of solvent is at least 80%. More preferably, the (preferably pharmaceutical) aqueous solution is water or saline.

In one embodiment, a pharmaceutical aqueous solution consists of water or a combination of water with salts like for example saline, Ringer's lactate solution and/or Plasma Lyte, wherein the amount of water of the total amount of solvent is at least 80%. More preferably, the (preferably pharmaceutical) aqueous solution is water or saline. In an even more preferred embodiment, a pharmaceutical aqueous solution consists of water.

A further preferred embodiment refers to a pharmaceutical aqueous solution comprising a solvent as described herein and a tonicity adjusting agent. Preferred tonicity adjusting agents for use in an aqueous solution are already described for the formulations according to the invention.

A further preferred embodiment refers to a pharmaceutical aqueous solution comprising a solvent as described herein and a tonicity adjusting agent and a buffer. Preferred tonicity adjusting agents and buffer systems for use in an aqueous solution are already described for the formulations according to the invention.

Preferably, the pH of a pharmaceutical aqueous solution is chosen so that the pH of the resulting liposomal formulation is between 5 and 8, preferably between 5 and 7.6; more preferably between 6 and 7.6 (e.g. either slightly acidic to neutral such as between 6.5 and 7 or between 7.2 and 7.6, such as between 7.3 and 7.5). The skilled person is aware that, depending on the preparation method according to the invention, a lyophilizate can already comprise a buffer system or such a buffer system is provided with a pharmaceutical aqueous solution. The skilled person can calculate the requirements on a pharmaceutical aqueous solution for preparing the final liposomal formulation without undue burden.

Preferred tonicity agents, buffer, solvent, phospholipid, phosphatidylglycerol, lipopeptide, Cholesterol (and derivatives thereof), saccharose components, ratios and amounts of any of the foregoing which can be used in a method of the invention are already described above for the formulations according to the invention.

Preferably, the ratios of the various components used in a method according to the invention are chosen by the skilled person to prepare a formulation with the requirements as described herein. The skilled person can easily calculate the required amounts and concentrations to prepare a formulation comprising
a) a phospholipid selected from the group consisting of phosphatidylcholine (PC), phosphatidylinositol (PI), phosphatidylserine (PS), phosphatidylethanolamine (PE), phosphatidic acid (PA), phosphatidylglycerol (PG)), or a derivative of any of the foregoing or mixtures thereof, in the range between 40% and 93% based on the total weight of a) to e);
b) Bulevirtide, in the range between 3% and 13% based on the total weight of a) to e) or Liraglutide in the range between 0.3% and 2% based on the total weight of a) to e);
c) cholesterol or a derivative thereof in the range between 0% and 14% based on the total weight of a) to e);
d) glycine, arginine, proline or any other amino acid known to be suitable as a bulking agent or a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture of any of the foregoing in the range between 0% and 35% based on the total weight of a) to e);
e) a tonicity adjusting agent which is not d) in the range between 0% and 35% based on the total weight of a) to e);
wherein the sum of a), b), c), d) and e) sums up to 100% and the combined amount of a) to e) is between 10% and 100% based on the total weight of the formulation and any preferred formulation.

In one preferred embodiment, in a method according to the invention a buffer must be present either in an aqueous phase or present in an aqueous solution.

In another preferred embodiment, in a method according to the invention the same or different buffers is/are present in the aqueous phase and present in an aqueous solution, preferably the same buffer.

In yet another preferred embodiment, in a method according to the invention a bulking agent is present in an aqueous phase.

In yet another preferred embodiment, in a method according to the invention a bulking agent and a buffer is present in an aqueous phase.

In yet another preferred embodiment, in a method according to the invention a tonicity adjusting agent is present in an aqueous phase.

In yet another preferred embodiment, in a method according to the invention a tonicity adjusting agent and a buffer is present in an aqueous phase.

In yet another preferred embodiment, in a method according to the invention a tonicity adjusting agent, a bulking agent and a buffer is present in an aqueous phase.

In yet another preferred embodiment, in a method according to the invention a tonicity adjusting agent is present in an aqueous solution.

In yet another preferred embodiment, in a method according to the invention a tonicity adjusting agent and a buffer is present in an aqueous solution.

### Kit

A further aspect of the invention refers to a kit comprising a formulation according to the invention and separated a pharmaceutical aqueous solution. For example, the formulation according to the invention is in one container while the pharmaceutical aqueous solution is in another container.

In one preferred embodiment, the formulation according to the invention is a lyophilized formulation.

In another preferred embodiment, the formulation according to the invention is a lyophilized formulation and a portion of said formulation in a container contains Bulevirtide (compound c)) in a concentration between 8 mg/176.6 mg total sum of components a) to e) and 19.4 mg /176.6 mg total sum of components a) to e) and the amount of Bulevirtide is between 5% and 11% compared to the total sum of a), b), c), d) and e) in the formulation. In one preferred embodiment, the weight of a portion of a formulation according to the invention in a container is between 100 mg and 250 mg.

In one preferred embodiment, the pharmaceutical aqueous solution is portioned in another container so that the weight of the portion of the pharmaceutical aqueous solution is calculated so that the amount of the sum of a), b), c), d) and e) in the resulting pharmaceutical liposomal formulation according to the invention after adding the portion of the pharmaceutical aqueous solution to the lyophilized formulation according to the invention (or vice versa) is between 20% and 2%, more preferably between 15% and 7% such as around 12%.

The mixing of the two components pharmaceutical aqueous solution and lyophilized formulation allows to prepare the pharmaceutical liposomal formulation shortly before administration to a patient.

While the described invention has been described with reference to the specific embodiments thereof it should be understood by those skilled in the art that various changes may be made, and equivalents may be substituted without departing from the true scope of the invention. In addition, many modifications may be made to adopt a particular situation, material, composition of matter, process, process step or steps, to the objective scope of the invention. All such modifications are intended to be within the scope of the claims appended hereto.

### FIGURES

FIG. 1 shows the process chart for a lyophilization process.

FIG. 2 shows the size distribution of observed vesicles depending on the ratio of the organic and aqueous phase (v/v).

FIG. 3 shows an average size of the liposome of 1.9 μm after reconstitution in 0.9% NaCl solution (D90=3.6 μm).

FIG. 4 shows an average size of the liposome of 0.2 μm after reconstitution in purified water (D90=2 μm).

FIG. 5 shows an RP-HPLC Chromatogram of the extra liposomal volume (un-encapsulated Bulevirtide).

FIG. 6 shows the force-displacement curve for Bulevirtide acetate liposomal formulation (1.5 ml) from a single-use 3 ml syringe with attached 27G×1″ canula.

Other aspects and advantages of the invention will be described in the following examples, which are given for purposes of illustration and not by way of limitation.

Each publication, patent, patent application or other document cited herein is hereby incorporated by reference in its entirety.

### Examples

### Substances and Materials

The following substances and materials were used (Table 1).

TABLE 1
Substances and materials

| | Lot-no.: | Manuf./Suppl. |
|---|---|---|
| Substances | | |
| Bulevirtide Acetate (Peptid 915207G) DS | Q33-Bulevirtide-47-200501 | Chengdu Shengnuo |
| | | Biopharm Co. Ltd. |
| Lipoid S100 | — | Lipoid |
| DOPG-Na 18:1/18:1, phosphatidylglycerol (PG) | — | Lipoid |
| tert-butanol (TBA) | 4323.1 | Carl Roth |
| cholesterol | 8866.1 | Carl Roth |
| Sodium acetate trihydrate, ≥99%, Ph. Eur., USP | 121162039 | Carl Roth GmbH |
| Acetic acid 100%, Emprove Expert, Ph. Eur., | K51921200 | Merck Chemicals |
| BP, JP, USP | | |
| Demineralised water, <0.2 μS/cm, TOC <10 | — | ProJect |
| ppb | | Pharmaceutics |
| Trifluoroacetic acid, ≥99.9%, for peptide | 039279763 | Carl Roth GmbH |
| synthesis | | |
| Acetonitrile, Chromosolv gradient grade | I2730 | Honeywell |
| Materials | | |
| Kinetex ® Pheny-Hexyl 2.6 μm; 4.6 × 150 mm | H19-347198 | phenomenex |
| column | | |
| filter 0.2 μm polyether sulfone Express Millex GP | SLGP033RS | Millipore |
| 33 mm 4.5 cm² | | |
| Stericup 0.22 μm Durapore membrane filter | MP183502G2 | Merck Millipore |
| Minisart 0.1 μm PES filter | 91076103 | Sartorius stedim |

The Following Equipment and Devices were Used:

### HPLC Equipment:

Ident. #: Sys 1
Manufacturer: Agilent Technologies (Santa Clara, California USA)
Type: 1260 Series

### Pilot Freeze Dryer (GT1):

Manufacturer: Hof Sonderanlagenbau (Lohra, Germany)
0.5 m2 shelf area

### Particle Sizer:

Manufacturer: Malvern (Malvern, GB)
Type: Mastersizer 2000

### DLS Plate Reader:

Manufacturer: Wyatt Technology Corporation (Santa Barbara, CA, USA)
Type: Dynapro plate reader II

### Additional Laboratory Equipment

Magnetic stirrer (IKA Werke)
Volumetric pipettes (Gilson)
Camera Equipment (Canon, EOS 600D with DGMacro 105 mm 1:2.8)
pH-meter (Mettler Toledo, SevenMulti with InLab Micro electrode)
Balance (Kern, EW6200-2 NM, ABJ-NM ABS3204N
Purified water supply (Siemens, Ultra Clean UV UF TM)
Vortex mixer (Scientific Industries Inc., Vortex Genie II)
Centrifuge: ThermoScientific, Heraeus Pico 17
Orbital shaker: Wisd Laboratory Instruments, WiseShake SHO-1D

The following protocol was used for RP-HPLC analysis (see Table 2):

TABLE 2
Chromatographic conditions for RP-HPLC

| Parameter | Value |
|---|---|
| Column Temperature | 35° C. |
| Sampler Temperature | 5 ± 2° C. |
| Wavelength | 210 nm (bandwidth 4 nm) |
| Pump Flow | 1.0 ml/min |
| Run Time | 40 min |
| Column | Agilent HC-C18, 5 μm, 4.6 × 250 mm |
| Mobile phase A | 50 mM potassium dihydrogen phosphate, pH 3.5 |
| Mobile phase B | Acetonitrile |
| Injection | Standard: 20 μg |

Gradient

| time [minutes] | % A | % B |
|---|---|---|
| 0 | 60 | 40 |
| 30 | 45 | 55 |
| 30.1 | 30 | 70 |
| 33 | 30 | 70 |
| 33.1 | 60 | 40 |
| 40 | 60 | 40 |

### Additional Laboratory Equipment

Magnetic stirrer (IKA Werke)
Volumetric pipettes (Gilson)
Camera Equipment (Canon, EOS 600D with DGMacro 105 mm 1:2.8)
pH-meter (Mettler Toledo, SevenMulti with InLab Micro electrode)
Balance (Kern, EW6200-2 NM, ABJ-NM ABS3204N
Purified water supply (Siemens, Ultra Clean UV UF TM)
Vortex mixer (Scientific Industries Inc., Vortex Genie II)
Centrifuge: ThermoScientific, Heraeus Pico 17
Orbital shaker: Wisd Laboratory Instruments, WiseShake SHO-1D

### Preparation of a Liposomal Formulation Via a Monophasic Nano-Dispersed System

| Liposomal composition per vial (after lyophilization) | | |
|---|---|---|
| fill volume | 1500.0 | mg |
| Bulevirtide-acetat (component b)) | 15.0 | mg |
| Lipoid S100 (component a)) | 100.5 | mg |
| DOPG-Na 18:1/18:1, phosphatidylglycerol (PG) (component a)) | 1.0 | mg |
| Cholesterol (component c)) | 15.1 | mg |
| Trehalose (component d)) | 45.0 | mg |
| total a) to d) | 176.6 | mg |
| total lipids | 116.6 | mg |
| 0.9% NaCl in water for reconstitution | 1323.4 | mg |
| % lipids (Cholesterol, PC (Lipoid S100), PG and Bulevirtide) | 8.8% | |
| after reconst. | | |

### Example 1: Preparation of a Nano-Dispersed System

Producing dispersions by utilizing TBA included the following steps:

An organic phase containing phophatidyl glycerol (PG), S100 (highly purified soy lecithin) and tert-butanol (TBA) was weighed and subsequently PG and S100 were dissolved in TBA by heating (70° C.) and agitation for approximately 40-80 min.

The organic phase was cooled down to ambient temperature, as soon as the ingredients were dissolved homogenously.

Aqueous phase: A 10 mM Sodium acetate buffer was adjusted to pH 5,5 with acetic acid. Trehalose (5%) was added and dissolved under agitation.

The aqueous solution was added in small portions to the organic phase under continuous stirring. First portions of aqueous solution were dissolved to a clear micellar system. After complete addition of the aqueous phase an opalescent monophasic nano-disperse system of low viscosity resulted.

Bulevirtide Acetate was added and loaded into the micellar system while gentle stirring.

The final bulk solution was sterile filtered through PVDF membrane filter of 200 nm nominal pore size.

| Preparation: | | |
|---|---|---|
| amount organic phase: | 50 | [g] |
| amount aqueous phase: | 50 | [g] |
| amount total bulk | 100 | [g] |
| Organic phase | | |
| Lipoid S100 | 6.700 | [g] |
| DOPG-Na 18:1/18:1, phosphatidylglycerol (PG) | 0.067 | [g] |
| Cholesterol | 1.01 | [g] |
| TBA | 42.228 | [g] |
| sum organic phase | 50.005 | g |
| Aqueous phase | | |
| 10 mM Acetate buffer pH 5.5 | 47 | [g] |
| Trehalose | 3 | [g] |
| sum aqueous phase | 50 | g |

### Example 2: Lyophilization

The formulation resulting from Example 1 was filled in sterilized glass vials (1.5 g in 2R vial).

Filled vials were stoppered in lyophilization position loaded into the freeze drier.

The following lyophilization cycle was performed (Table 3).

TABLE 3
Program of the lyophilization process

| | | | Ice | | | |
|---|---|---|---|---|---|---|
| | | Shelf | condenser | Pressure | | Cumulative |
| Step | | temperature | temperature | (MKS) | Time step | time |
| # | Description | [° C.] | [° C.] | [mbar] | [h:min] | [h:min] |
| 1 | Loading | 20 | — | 1000 | 00:01 | 00:01 |
| 2 | Freezing Ramp | −45 | — | 1000 | 01:00 | 01:01 |
| 3 | Freezing | −45 | — | 1000 | 03:00 | 03:01 |
| 4 | Vacuum Adjustment | −45 | −70 | 0.1 | 00:30 | 03:31 |
| 5 | Primary Drying Ramp | −10 | −70 | 0.1 | 03:00 | 06:31 |
| 6 | Primary Drying | −10 | −70 | 0.1 | 16:30 | 23:01 |
| 7 | Secondary Drying Ramp | 35 | −70 | 0.1 | 01:00 | 24:01 |
| 8 | Secondary Drying | 35 | −70 | 0.1 | 04:00 | 28:01 |

### The Following Apparatus was Used:

Pilot freeze dryer (GT3) (Hof Sonderanlagenbau (Lohra, Germany)), 0.25 m2 shelf area, 5 kg ice condenser capacity.

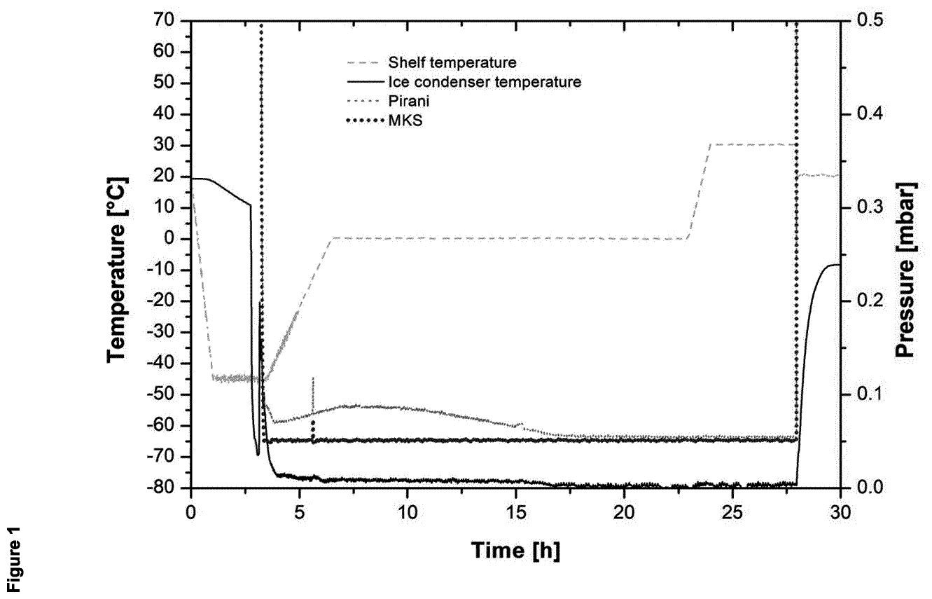

The process was monitored via online data acquisition. The process chart (see FIG. 1) showed that the lyophilization process was completed successfully.

### Example 3: Preparation of Liposomal Formulation

The lyophilisate was reconstituted with either 1.5 ml purified water or 1.5 ml 0.9% sodium chloride solution. Reconstitution of the lyophilizate was fast and spontaneous within 10 seconds. The obtained dispersion was homogenized by gently swirling within 5 minutes or vortex mixing.

For both cases visually different liposomal dispersions were obtained. Whereas the lyophilizate reconstituted with purified water showed a higher transparency and opalescence, indicating particles of sub-micron size, the one reconstituted with purified water showed a higher turbidity and more milky appearance. More specifically, whereas the lyophilizate reconstituted with purified water showed a higher transparency and opalescence, indicating particles of sub-micron size, the one reconstituted with saline showed a higher turbidity and more milky appearance indicating multilamellar liposomes having larger diameter. Thus, it is advantageous, especially for subcutaneous depot formulations, to reconstitute the lyophilizates using saline.

Both reconstitution variants were analyzed using a MALS particle sizer.

### Example 4: Vesicle/Liposome Size Distribution

MALS (multiangle light scattering): The liposome/vesicle size was determined using a Malvern, Mastersizer 2000 with hydro 2000 μP sample cell instrument (MALS) equipped with liquid sample cell. The sample cell was filled with the dispersant (˜ 18 mL, 0.9% sodium chloride or purified water) and air bubbles were removed through increasing circulation pump speed. The instrument was blanked. Sample was added until sufficient signal absorption was reached. The measurement was started, and three subsequent measurements were taken. The result was calculated from the summarized data.

DLS (dynamic light scattering): The liposome/vesicle size distribution was also determined using a Dynapro Plate Reader (Wyatt Technology) instrument. A sample (30 μL) was filled into a 96 well plate with transparent bottom and the plate was transferred into the DLS plate reader, three wells were filled per sample. The measurement was started. The temperature was set to 25° C. Acquisition time: 5 s, five acquisitions were taken per well. The mass weighted mean radius of the observed particles was calculated.

### 4.1 Vesicle (Micelle) Sizes in the Monophasic Nano-Dispersed System

Different ratios of the organic and aqueous phase were analyzed by dynamic light scattering, to determine the size of the formed vesicles.

Tested ratios were org. phase:aqueous phase 1:1, 1:3 and 1:5.

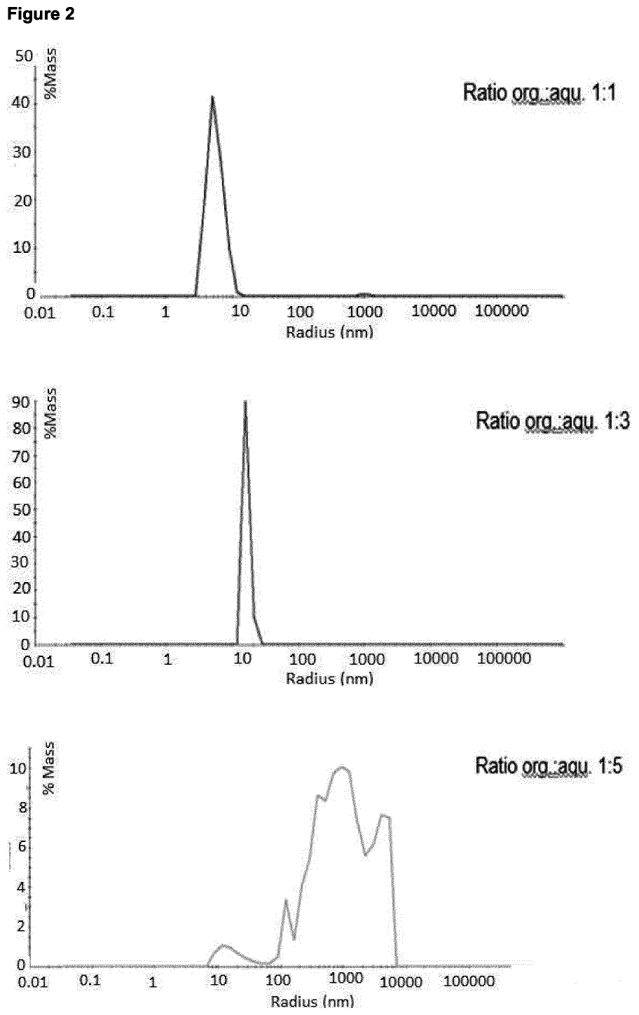

A ratio of 1:1 led to a clear solution without any turbidity. Extremely small particles in low number could be detected. The mean size was around 5 nm (3 nm to 10 nm, see FIG. 2). Without being bound to this explanation, the high ratio of organic solvent seems to lead to a predominantly molecular solution of the components within the solvent mixture.

Within a solvent mixture ratio of 1:3 the number and size of the observed particles increased substantially to about 15 nm mean size (10 nm to 20 nm, see FIG. 2). A micellar solution was formed showing the typical size and homogeneity of micelles. The mixture showed a nearly clear appearance with slight Tyndall effect and was freely filterable through a sterile filter with 0.22 μm nominal pore size.

At a solvent mixture ratio of 1:5 the resulting preparation was turbid. Size distribution measurement by DLS revealed a broad inhomogeneous spectrum of vesicles having a mean size of about 1 μm which is typical for multilamellar liposomes. This liposomal dispersion can be filtered through a membrane sterile filter with 0.22 μm nominal pore size only by applying high pressure, leading to deformation of the vesicles. FIG. 2 shows the size distribution of observed vesicles depending on the ratio of the organic and aqueous phase (v/v).

### 4.2 Liposome Sizes in the Liposomal Formulation (Reconstituted Lyophilizate)

The following size distributions were observed:

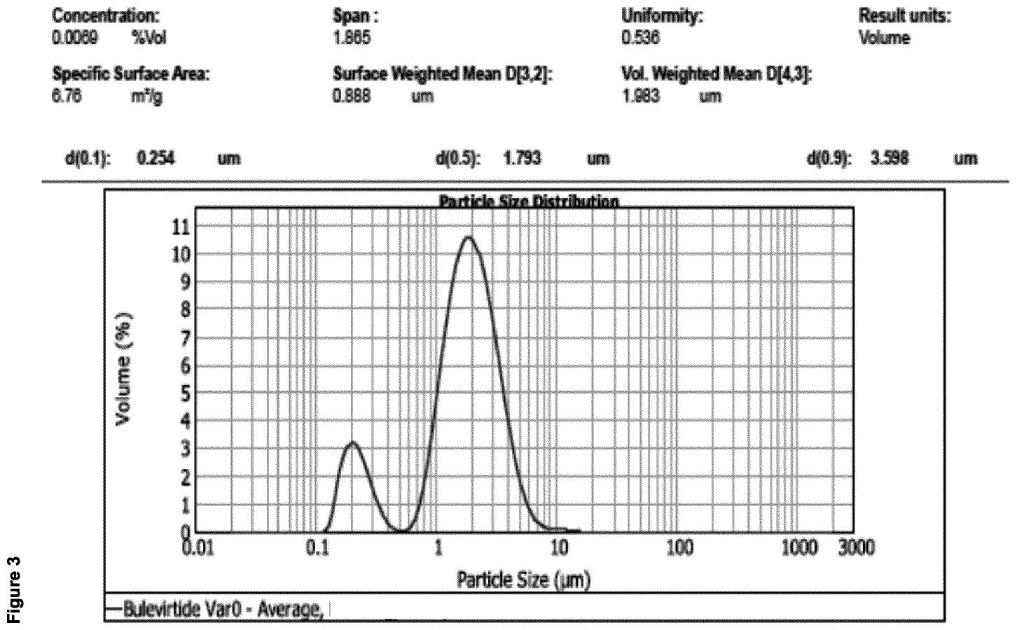

Reconstitution in 0,9% NaCl solution leads to an average size of the liposome of 1.9 μm (D90=3.6 μm, see FIG. 3)

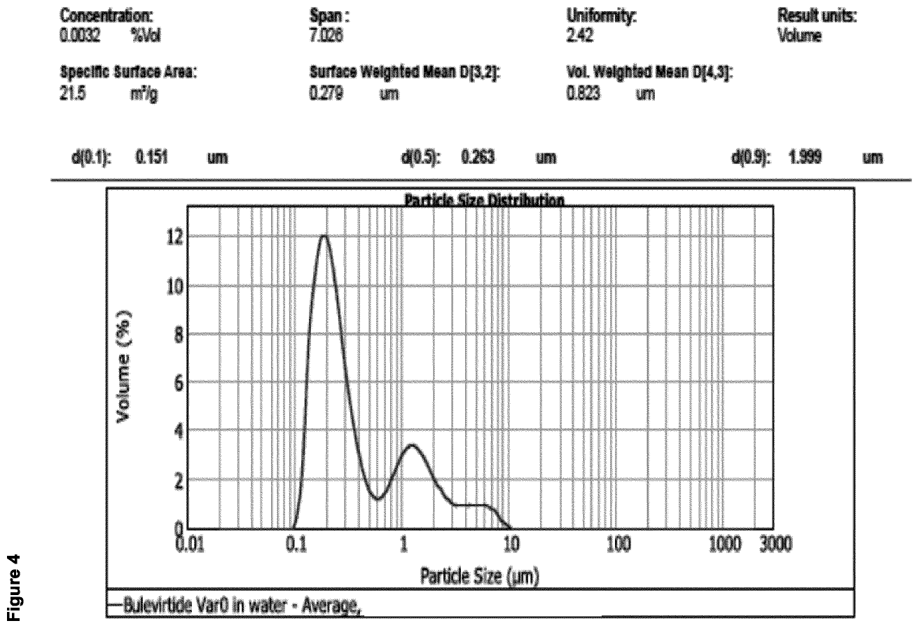

Reconstitution in purified water leads to an average size of the liposome of 0.2 μm (D90=2 μm, see FIG. 4).

The measured values supported the visual impression of the dispersions: Whereas larger liposomes were formed upon reconstitution in sodium chloride solution (main fraction around 1.9 μm with a D90 of 3.6 μm) liposomes of smaller size were observed upon reconstitution in purified water (main fraction around 0.2 μm with a D90 of 2 μm). As discussed above, those larger (multilamellar) liposomes are preferred as providing higher drug loads.

### Example 5: Liposomal Encapsulation of Bulevirtide

Determination of content and purity of Bulevirtide and the drug product.

The liposomal encapsulation of Bulevirtide was studied by determining the amount of free (unencapsulated) Bulevirtide.

Lyophilized samples from Example 2 were reconstituted with 1.5 ml 0.9% (w/w) NaCl solution or water. Samples for determining the liposomal encapsulation of Bulevirtide were mixed and left for 20 minutes to allow complete hydrazination and liposome formation. Subsequently, reconstituted samples were diluted 1:1 with 0.9% sodium chloride solution and mixed thoroughly. Samples were centrifuged at 21460×g to separate the lipid phase from the solvent. The supernatant was collected and analyzed by RP-HPLC:

RP-HPLC method was used as provided from the manufacturer of the drug substance, Chengdu Shengnuo Biopharm Co. Ltd. The chromatographic conditions were used for the analysis as outlined in Table 2.

Samples were reconstituted with 1.5 mL 0.9% (w/w) sodium chloride solution. Samples were mixed well and left on the lab bench for 20 minutes to allow complete hydrazination and liposome formation. Subsequently, reconstituted samples were diluted 1:1 with 0.9% sodium chloride solution and mixed thoroughly. Samples were centrifuged at 21460×g to separate the lipid phase from the solvent. The supernatant was collected and analyzed.

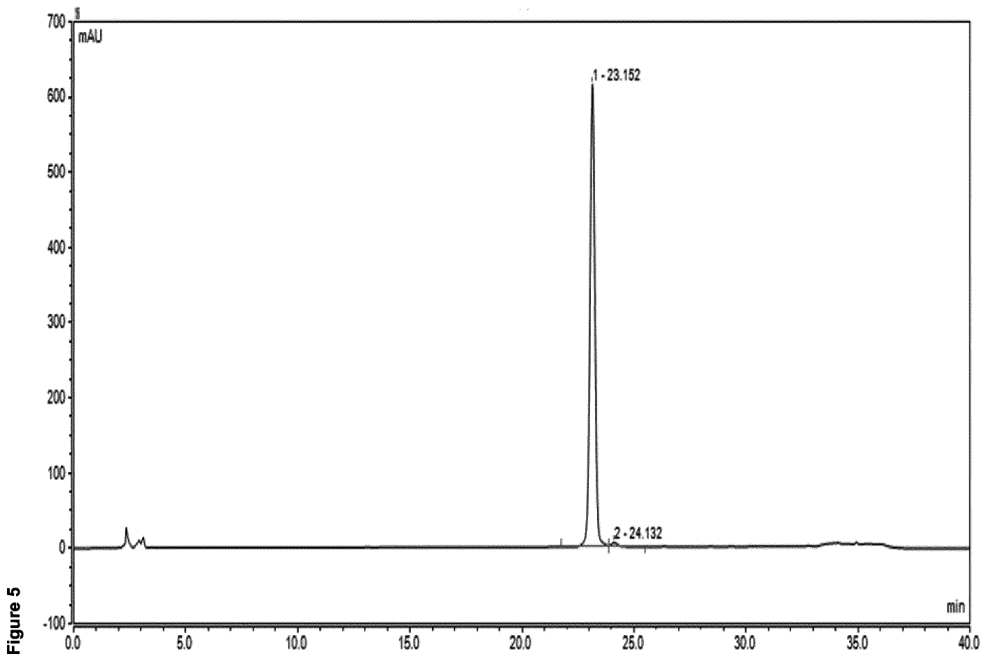

Results are reported as is mg/ml using a DS calibration standard and the peak area of Bulevirtide from the chromatogram obtained with the sample preparation. FIG. 5 shows an RP-HPLC Chromatogram of the extra liposomal volume (un-encapsulated Bulevirtide).

Small amounts of Bulevirtide could be detected in the supernatant. By comparison to a calibration standard, the amount of free Bulevirtide acetate was calculated to 847 μg (per vial), which corresponds to an encapsulation efficiency of 94%. In a second vial of the same preparation, an encapsulation efficiency of 96% (584 μg per vial) was determined.

### Example 6: Push-Out Force

After reconstitution of a lyophilizate with water or 0,9% NaCl, respectively, the liposome dispersion was sucked into a standard single-use 3 mL PP syringe, a 27G x 1″ canula was attached and remaining air was removed from the syringe. The content of the syringes was dispensed with a defined traverse speed using a force/displacement measuring device (Thümler, Z3).

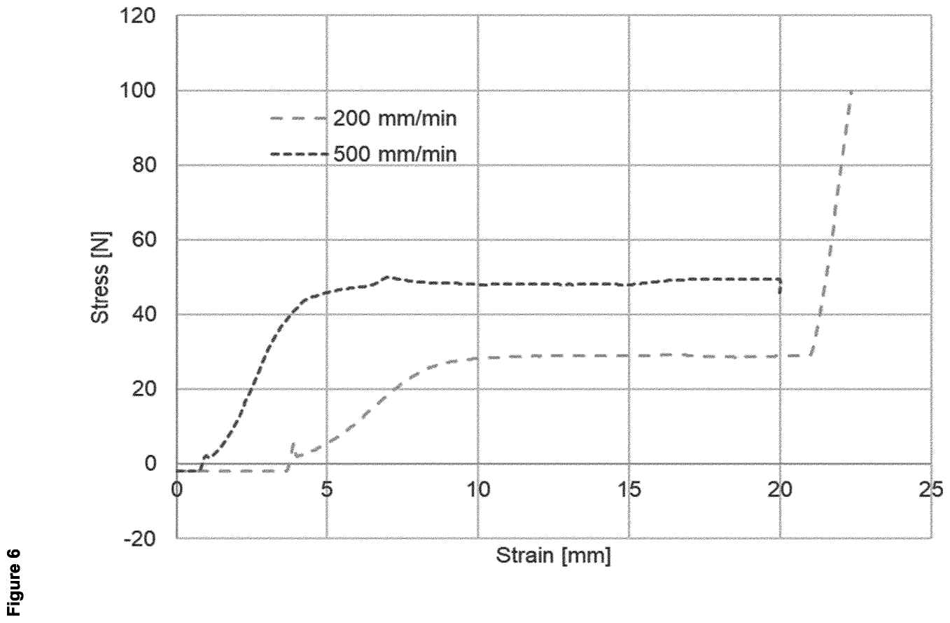

The results are shown in FIG. 6. The figure shows the force-displacement curve for Bulevirtide acetate liposomal formulation (1.5 ml) from a single-use 3 ml syringe with attached 27G x 1″ canula. Strain 1 of FIG. 1: speed 200 mm/min; Strains 2 and 3 of FIG. 5: speed 500 mm/min.

Evaluation of the push-out force showed that a moderate force of 30 to 50 N is sufficient to dispense the liposomal Bulevirtide formulation.

1. A method for preparing a formulation comprising the steps of
i) providing an organic phase comprising
one or more phospholipid(s) selected from the group consisting of phosphatidylcholine (PC), phosphatidylinositol (PI), phosphatidylserine (PS), phosphatidylethanolamine (PE), phosphatidic acid (PA), phosphatidylglycerol (PG) or a derivative of any of the foregoing or a combination of any of the foregoing; and
optionally Cholesterol or a derivative thereof; and
at least one organic solvent;
ii) providing an aqueous phase comprising an aqueous medium; and
optionally a pharmaceutically acceptable buffer, and
optionally a bulking agent selected from the group consisting of glycine, arginine, proline or any other amino acid known to be suitable as a bulking agent, a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture of any of the foregoing, and
optionally a pharmaceutically acceptable tonicity adjusting agent which is not a bulking agent selected from the group consisting of glycine, arginine, proline or any other amino acid known to be suitable as a bulking agent, a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture of any of the foregoing,
wherein the pH of the aqueous phase is between 3 and 9;
iii) Combining the organic phase and the aqueous phase, wherein the mixing ratio of the organic phase and the aqueous phase is between 10:1 (v/v) and 1:10 (v/v) resulting in a combined organic and aqueous phase; wherein said at least one organic solvent and the aqueous phase form a monophasic mixture, preferably a freezable and sublimable monophasic mixture;
iv) adding a lipopeptide either to the organic phase of step i) and then mixing the organic phase with the lipopeptide with the aqueous phase as described in step iii), or to the aqueous phase of step ii) and then mixing the aqueous phase with the lipopeptide with the organic phase as described in step iii), or to the combined phases of step iii), resulting in a lipopeptide formulation, wherein said lipopeptide formulation so obtained is a monophasic nano-disperse system.

2. The method according to claim 1, wherein the at least one organic solvent is selected from the group consisting of anisole, ethylacetate, 1,4-dioxane, dimethylcarbonate, dimethylsulfoxide, glycofurol, N,N-dimethylacetamide, N,N-dimethylformamide, N-methyl-2-pyrrolidone (NMP), isopropylideneglycerol, an alcohol, preferably an alcohol selected group consisting of 1-butanol, 2-butanol and tert-butanol, acetic acid, ethyl lactate (ethyl 2-hydroxypropanoate), acetonitrile, or a combination of any of the foregoing, preferably the at least one organic solvent is selected from the group consisting of an alcohol, preferably tert-butanol, anisole (phenoxymethane), dimethylsulfoxide, 1,4-dioxane and dimethylcarbonate and a combination thereof.

3. The method according to claim 1, wherein the derivative of any of the foregoing is selected from the group consisting of: DLPA, DMPA, DPPA, DSPA, POPA, POPA, DEPA, HSPA, HEPA, DLPC, DMPC, DPPC, DSPC, DOPC, POPC, DEPC, HSPC, HEPC, DLPE, DMPE, DPPE, DSPE, POPE, POPE, DEPE, HSPE, HEPE, DLPG, DMPG, DPPG, DSPG, POPG, POPG, DEPG, HSPG, HEPG, DLPI, DMPI, DPPI, DSPI, POPI, POPI, DEPI, HSPI, HEPI, DLPS, DMPS, DPPS, DSPS, POPS, POPS, DEPS, HSPS, HEPS, a PEGylated form of any of the foregoing and a salt of any of the foregoing.

4. The method according to claim 1, wherein the organic solvent is tert-butanol.

5. The method according to claim 1, wherein the lipopeptide is Bulevirtide or Liraglutide.

6. The method according to claim 1, wherein the phospholipid comprises PC.

7. The method according to claim 1, wherein the pH of the aqueous phase is between 5 and 7.5.

8. The method according to claim 1, further comprising sterile filtering of the monophasic nano-disperse system.

9. The method according to claim 1 further comprising a step v) of lyophilizing the formulation (monophasic nano-disperse system) resulting from step iv) which results in a lyophilizate.

10. The method according to claim 9 further comprising a rehydration step of mixing the lyophilizate obtained in step v) with an aqueous solution resulting in a liposomal formulation.

11. The method according to claim 10, wherein the D90 of the liposomes of the liposomal formulation resulting from the rehydration step (reconstitution) is between 1 μm and 4.5 μm.

12. The method according to claim 11, wherein the aqueous solution used for mixing/reconstituting is an aqueous NaCl solution wherein the amount of NaCl is between 8 g/l and 10 g/l, preferably between 8.8 g/l and 9.2 g/l, more preferably 9 g/l.

13. The method according to claim 1, wherein the D90 of the micelles of the monophasic nano-disperse system is 60 nm or less.

14. (canceled)

15. A formulation, wherein the formulation is a monophasic nano-disperse system comprising
a) a phospholipid selected from the group consisting of phosphatidylcholine (PC), phosphatidylinositol (PI), phosphatidylserine (PS), phosphatidylethanolamine (PE), phosphatidic acid (PA), phosphatidylglycerol (PG), or a derivative of any of the foregoing or mixtures thereof, in the range between 40% and 97% based on the total weight of a) to e);
b) Bulevirtide, in the range between 3% and 13% based on the total weight of a) to e) or Liraglutide in the range between 0.3% and 2% based on the total weight of a) to e);
c) cholesterol or a derivative thereof in the range between 0% and 14% based on the total weight of a) to e);
d) glycine, arginine, proline or any other amino acid known to be suitable as a bulking agent or a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture of any of the foregoing in the range between 0% and 35%, preferably between 15% and 35% based on the total weight of a) to e);
e) a tonicity adjusting agent which is not d) in the range between 0% and 35% based on the total weight of a) to e);
wherein the sum of a), b), c), d) and e) always sums up to 100% and the combined amount of a) to e) is between 10% and 100% based on the total weight of the formulation.

16. The formulation according to claim 15, wherein the formulation is a lyophilized formulation.

17. A method of preparing a pharmaceutical liposomal formulation, comprising:
providing a formulation of claim 16; and reconstituting the formulation with an aqueous solution, wherein the pharmaceutical liposomal formulation comprises
a) a phospholipid selected from the group consisting of phosphatidylcholine (PC), phosphatidylinositol (PI), phosphatidylserine (PS), phosphatidylethanolamine (PE), phosphatidic acid (PA), phosphatidylglycerol (PG), or a derivative of any of the foregoing or mixtures thereof in the range between 40% and 75% based on the total weight of a) to e);
b) Bulevirtide, in the range between 3% and 13% based on the total weight of a) to e) or Liraglutide in the range between 0.3% and 2% based on the total weight of a) to e);
c) cholesterol or a derivative thereof in the range between 4% and 14% based on the total weight of a) to e);
d) glycine, arginine, proline or any other amino acid known to be suitable as a bulking agent or a saccharide component selected from the group consisting of sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture thereof in the range between 15% and 35% based on the total weight of a) to e);
e) a tonicity adjusting agent which is not d) in the range between 0.1% and 10% based on the total weight of a) to e);
wherein the sum of a), b), c), d) and e) sums up to 100% and the combined amount of a) to e) is between 10% and 50% based on the total weight of the formulation further comprising a solvent and a tonicity adjusting agent.

18. A kit comprising a formulation according to claim 15 in a container and a pharmaceutical aqueous solution in a second container.

19. The method according to claim 1, wherein the derivative of cholesterol is selected from the group consisting of cholesteryl sulfate, a salt of cholesteryl sulfate, cholesteryl hemisuccinate, cholesteryl succinate, cholesteryl oleate, cholesterol-PEG, coprostanol, cholestanol, cholestane, cholic acid, cortisol, corticosterone, hydrocortisone and calciferol.

20. A kit comprising a formulation according to claim 16 in a container and a pharmaceutical aqueous solution in a second container.

21. The formulation according to claim 15, wherein the derivative of cholesterol is selected from the group consisting of cholesteryl sulfate, a salt of cholesteryl sulfate, cholesteryl hemisuccinate, cholesteryl succinate, cholesteryl oleate, cholesterol-PEG, coprostanol, cholestanol, cholestane, cholic acid, cortisol, corticosterone, hydrocortisone and calciferol.

22. The formulation according to claim 16, wherein the derivative of cholesterol is selected from the group consisting of cholesteryl sulfate, a salt of cholesteryl sulfate, cholesteryl hemisuccinate, cholesteryl succinate, cholesteryl oleate, cholesterol-PEG, coprostanol, cholestanol, cholestane, cholic acid, cortisol, corticosterone, hydrocortisone and calciferol.
